(19) United States
(12) Patent Application Publication
(10) Pub. No.: US 2025/0090480 A1
(43) Pub. Date: Mar. 20, 2025

(54) UNIQUE ANALOGS OF NATURAL LIGNANS USEFUL FOR TREATING TRIPLE NEGATIVE BREAST CANCER

(71) Applicant: University of Kentucky Research Foundation, Lexington, KY (US)

(72) Inventors: Bert C. Lynn, Nicholasville, KY (US); Samuel G. Awuah, Lexington, KY (US); Alyson M. Ackerman, Lexington, KY (US)

(21) Appl. No.: 18/889,177

(22) Filed: Sep. 18, 2024

Related U.S. Application Data
(60) Provisional application No. 63/538,942, filed on Sep. 18, 2023.

(51) Int. Cl. A61K 31/09; A61K 45/06; A61P 35/00; C07C 41/09; C07C 43/23

(52) U.S. Cl. CPC A61K 31/09; A61K 45/06; A61P 35/00; C07C 41/09; C07C 43/23

(57) ABSTRACT

The presently-disclosed subject matter provides compounds and compositions comprising unique analogs of lignans, methods of making such compounds, and methods of using such compounds.

### RELATED APPLICATIONS

This application claims priority from U.S. Provisional Application Ser. No. 63/538,942 filed Sep. 18, 2023, the entire disclosure of which is incorporated herein by this reference.

### GOVERNMENT INTEREST

This invention was made with government support under grant number CA258421-01 awarded by the National Institutes of Health, grant numbers OIA1632854 and 1922694 awarded by the National Science Foundation. The government has certain rights in the invention.

### TECHNICAL FIELD

The presently-disclosed subject matter relates to compounds and compositions comprising unique analogs of lignans, methods of making such compounds and compositions, and methods of treatment.

### INTRODUCTION

Lignans are naturally occurring phenolic dimers and trimers, which share common monomers with lignin, a rigid structural component of plant cell walls.^{[1]} Although bioactive properties of lignans have been reported and implicated in traditional medicine, a systematic lignan-based natural product probe/drug discovery is non-existent.^{[2,3]} The historical role of natural products (NPs) for use as therapeutic agents against cancer and infectious diseases is significant.^{[4]} Previous NP scaffolds have identified distinct biological targets and treatment options with uniquely tolerable and effective properties.^{[5]} The structural, stereochemical complexity and diversity of natural products present innovative opportunities for disease treatment, but not without challenges. The reduction of natural product drug discovery campaigns is associated with insufficient natural product material for isolation; physicochemical barriers; laborious structure-activity relationship for lead optimization; and difficulty with dissecting mechanism of action.^{[6-10]} Leveraging streamlined diversification synthetic strategies and small-molecular weight natural products, such as lignans, has the potential to transform NP drug discovery.

Traditional Chinese medicine is used as an alternative or complementary treatment for various cancers and diseases, boasting of reduced side effects and prolonged survival times.^{[11]} Although it is not recommended as the primary course of treatment, there is mounting evidence to support elements of efficacy, specifically with regards to Chinese herbal medicine.^{[12]} Plants that have been used medicinally for centuries are under scrutiny to pinpoint their active ingredients and maximize their potential. One such plant is the Asian's Lizard Tail (Saururus chinensis), a flowering herb whose leaves have been used for centuries to treat various inflammatory conditions.^{[13]} A recent publication isolated the lignan compound machilin D from this plant and determined it to be cytotoxic against breast cancer stem cells and well tolerated in cancer mouse models.^{[14]} This adds to a host of evidence that this compound, and its threomer counterpart machilin C, possess biological activity,^{[15-17]} and highlights a novel treatment for breast cancer.

Machilins C and D were first identified and named in 1987 as components of the extract from Machilus thunbergii, the Japanese bay tree.^{[18]} Since that initial study, several additional reports have been published exalting the diverse therapeutic benefits of both compounds as well as whole extracts from both Saururus chinensis^{[36]} and Machilus thunbergii[^{37]}.

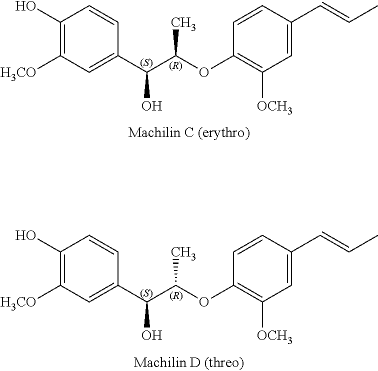

These agents have been investigated for the treatment of diabetes,^{[19]} treatment of Alzheimer's Disease,^{[20]} promotion of vasorelaxation,^{[21, 22]} anti-oxidant properties,^{[18, 23, 24]} and anti-inflammatory effects.^{[14, 17, 25}] Current studies have posited machilins C and D as anticancer agents in the treatment of stomach,^{[16]} oral,^{[26]} breast,^{[14]} and blood^{[27, 28]} cancers. Potential mechanisms of action include promotion of apoptosis and autophagy,^{[26]} inhibition of DNA topoisomerases I and II,[^{29]} prevention of nitric oxide production,^{[27, 28]} and blocking of interleukin IL-6 and IL-8 signaling.^{[14]}

Breast cancer is the most common form of cancer in women and the second leading cause of their cancer related death. Triple-negative breast cancer (TNBC) is a type of breast cancer characterized by its lack of expression of all three of estrogen receptors, progesterone receptors, and human epidermal growth factor receptor 2 (HER-2).^{[30]} TNBC mostly occurs in younger women (premenopausal) and represents ˜15% of all breast cancers. TNBC is highly invasive and, as compared with other types of breast cancer, it is associated with higher recurrence and shorter survival time.

In non-TNBCs, the estrogen, progesterone, and HER-2 receptors are leveraged as drug targets to develop selective treatments with reduced side effects.^{[31-33]} The absence of these targets relegates TNBC treatment to conventional therapies including chemotherapy, radiation, and tumor/tissue resection.^{[34]} Examples of some current treatment approaches for TNBC include platinum agents for a subset of patients with BRCA mutations; taxanes; anthracycline, and more recently a combination therapy involving Atezolizumab (PD-L1 inhibitor) and Nab-paclitaxel for metastatic TNBC. Nevertheless, these chemotherapeutic strategies are limited by acquired or intrinsic resistance, lack of potency, cross-resistance, and devastating side effects that greatly diminish quality of life.

Accordingly, there remains in the art an urgent need for additional chemotherapy and targeted therapy options having unique mechanisms for use in the treatment of TNBC.

### SUMMARY

The presently-disclosed subject matter meets some or all of the above-identified needs, as will become evident to those of ordinary skill in the art after a study of information provided in this document.

The presently-disclosed subject matter includes compounds and compositions comprising unique analogs of lignans, methods of making such compounds and compositions, and methods of treating cancer using such compounds and compositions.

Disclosed herein are unique compounds having superior anticancer activity as compared to machilin C/D. Also disclosed herein is a streamlined synthetic protocol to generate lignan-based drug candidates as anti-cancer agents and identify their structure-activity relationship (SAR). Evaluation of these compounds in 2D monolayer and 3D mammosphere assays shows anticancer activity against triple negative breast cancer (TNBC).

The presently-disclosed subject matter includes a compound of the following formula:

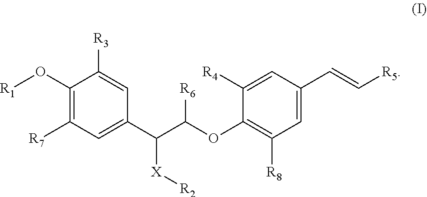

X is selected from the group consisting of O, NH, and S; R₁ is selected from the group consisting of H, lower alkyl, and acetyl; R₂ is selected from the group consisting of H, lower alkyl, benzyl, ethyl 2-hydroxy, cyclohexyl, methylcyclohexyl and N,N-dimethylaminoethyl, so long as R₂ is not H when X is O; R₃ is selected from the group consisting of H, methoxy, fluorine, and cyano; R₄ is selected from the group consisting of H, hydroxyl, methoxy, fluorine, and cyano; R₅ is selected from the group consisting of H, methyl, hydroxyl, hydroxymethyl, methoxy, methoxymethyl, and acetate; R₆ is selected from the group consisting of methyl, hydroxyl, hydroxymethyl, and methoxymethyl; R₇ is selected from the group consisting of H, hydroxyl, and methoxy; and R₈ is selected from the group consisting of H, hydroxyl, and methoxy.

The presently-disclosed subject matter further includes compositions comprising a compound as disclosed herein and a pharmaceutically-acceptable carrier.

The presently-disclosed subject matter further includes methods of using the compounds and compositions as disclosed herein. A method for conferring anti-cancer activity to a cancer cell, comprises contacting the cancer cell with an effective amount of a compound or composition as disclosed herein.

The presently-disclosed subject matter further includes methods of making the compounds as disclosed herein.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 is a schematic illustrating the increased efficacy of the compounds of the presently-disclosed subject matter for conferring anti-cancer activity, as compared to the naturally-occurring lignan compounds machilin C and D, and the increased efficiency of method of the presently-disclosed subject matter for synthesizing naturally-occurring lignan compounds and compounds of the presently-disclosed subject matter, as compared to methods that of isolating active compounds from plants.

FIG. 2A-2C. Cell viability upon treatment with compound 7 in TNBCs at 24 h by MTT (FIG. 2A). Cell viability upon treatment with cisplatin or doxorubicin in MDA-MB-231 (FIG. 2B) and MDA-MB-468 (FIG. 2C) at 24 h by MTT

FIG. 3A-3B. C-300 inhibits colony formation in MDA-MB-231 after 7 days (FIG. 3A). Dose-dependent depletion of c-MYC by C-300 in MDA-MB-231 after 24 h (FIG. 3B).

FIG. 4. Change in body weight. C57BL6 mice were injected with compound 7 (C-300) at 50 mg/kg or 100 mg/kg 3× a week for 2-week.

FIG. 5A. IC₅₀ (±SEM) values as calculated by MTT assay in three TNBC cell lines.

FIG. 5B. Comparison bar graph of IC₅₀ values for compounds 1-8.

FIG. 5C. Dose-Response curve of compound 1 (machilin C/D) in TNBC cell lines MDA-MB-231, MDA-MB-468, SUM159.

FIG. 5D. Dose-Response curve of compound 2 in TNBC cell lines MDA-MB-231, MDA-MB-468, SUM159.

FIG. 5E. Dose-Response curve of compound 3 in TNBC cell lines MDA-MB-231, MDA-MB-468, SUM159.

FIG. 5F. Dose-Response curve of compound 4 in TNBC cell lines MDA-MB-231, MDA-MB-468, SUM159.

FIG. 5G. Dose-Response curve of compound 5 in TNBC cell lines MDA-MB-231, MDA-MB-468, SUM159.

FIG. 5H. Dose-Response curve of compound 6 in TNBC cell lines MDA-MB-231, MDA-MB-468, SUM159.

FIG. 5I. Dose-Response curve of compound 7 in TNBC cell lines MDA-MB-231, MDA-MB-468, SUM159.

FIG. 5J. Dose-Response curve of compound 8 in TNBC cell lines MDA-MB-231, MDA-MB-468, SUM159.

FIG. 6A. HPLC chromatograms of fractions obtained from a manual silica column.

FIG. 6B. Segment of ¹H NMR spectrum comparing J-coupling values for protons 8 and 9 on compounds 7A and 7B.

FIG. 6C. Comparison of IC₅₀ values for compounds 7A and 7B in three TNBC cell lines (***P=0.003, ****P<0.0001).

FIG. 6D. Dose-Response curve of compound 7A in TNBC cell lines MDA-MB-231, MDA-MB-468, SUM159.

FIG. 6E. Dose-Response curve of compound 7B in TNBC cell lines MDA-MB-231, MDA-MB-468, SUM159.

FIG. 7A. Colonogenic assay images in triplicate for compounds 1, 7A, and 7B.

FIG. 7B. Select mammosphere images taken over period of 6 days for compounds 1, 7A, and 7B.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

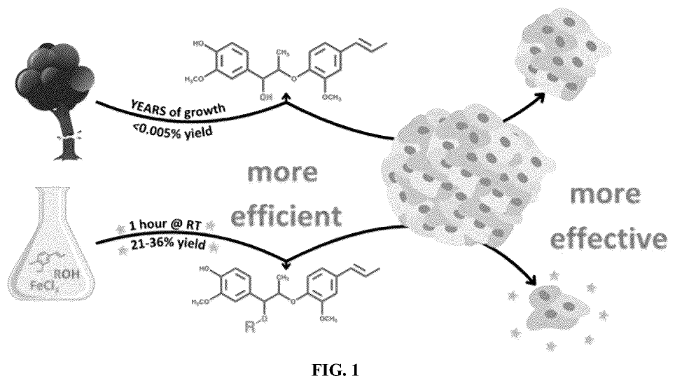

The presently-disclosed subject matter includes compounds and compositions comprising unique analogs of lignans, methods of making such compounds and compositions, and methods of treating cancer using such compounds and compositions. As disclosed herein, the presently-disclosed subject matter provides unique compounds that are more effective for conferring anti-cancer activity as compared to the naturally-occurring lignan compounds machilin C and D (FIG. 1). The presently-disclosed subject matter also provides a method of synthesizing naturally-occurring lignan compounds and the unique compounds as disclosed herein, which is more efficient as compared to methods that of isolating active compounds from plants (FIG. 1).

The presently-disclosed subject matter includes a compound of the following formula:

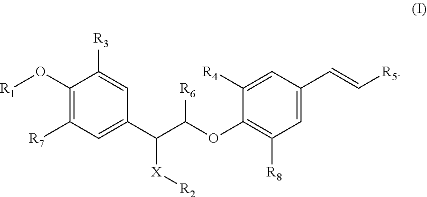

X is selected from the group consisting of O, NH, and S; R₁ is selected from the group consisting of H, lower alkyl, and acetyl; R₂ is selected from the group consisting of H, lower alkyl, benzyl, ethyl 2-hydroxy, cyclohexyl, methylcyclohexyl and N,N-dimethylaminoethyl, so long as R₂ is not H when X is O; R₃ is selected from the group consisting of H, methoxy, fluorine, and cyano; R₄ is selected from the group consisting of H, hydroxyl, methoxy, fluorine, and cyano; R₅ is selected from the group consisting of H, methyl, hydroxyl, hydroxymethyl, methoxy, methoxymethyl, and acetate; R₆ is selected from the group consisting of methyl, hydroxyl, hydroxymethyl, and methoxymethyl; R₇ is selected from the group consisting of H, hydroxyl, and methoxy; and R₈ is selected from the group consisting of H, hydroxyl, and methoxy.

In some embodiments of the compound of Formula I, X is O and R₂ is methyl, ethyl, propyl, butyl, isopropyl, isobutyl, or isopentyl. In some embodiments of the compound of Formula I, X is NH and R₂ is H, methyl, ethyl, propyl, butyl, isopropyl, isobutyl, or isopentyl. In some embodiments of the compound of Formula I, X is S and R₂ is H, methyl, ethyl, propyl, butyl, isopropyl, isobutyl, or isopentyl.

In some embodiments of the compound of Formula I, R₁ is selected from the group consisting of H, methyl, and acetyl. In some embodiments of the compound of Formula I, R₃ is H or methoxy. In some embodiments of the compound of Formula I, R₄ is H or hydroxyl. In some embodiments of the compound of Formula I, R₅ is selected from the group consisting of methyl, hydroxymethyl, methoxy, methoxymethyl. In some embodiments of the compound of Formula I, R₆ is selected from the group consisting of methyl, hydroxyl, hydroxymethyl, and methoxymethyl. In some embodiments of the compound of Formula I, R₇ is selected from the group consisting of H, hydroxyl, and methoxy. In some embodiments of the compound of Formula I, R₈ is selected from the group consisting of H, hydroxyl, and methoxy.

In some embodiments of the compound of Formula I, R1 is H; R3 is H or methoxy; R4 is H or hydroxyl; R5 is methyl, hydroxymethyl, methoxy, or methoxymethyl; R6 is methyl; R7 is methoxy; and R8 is methoxy. In some embodiments of the compound of Formula I, R1 is H; R3 is H; R4 is H; R5 is methyl; R6 is methyl; R7 is methoxy; R8 is methoxy; and X is O.

In some embodiments, diastereomers of the compound are provided. In some embodiments an erythromer is provided selected from the following:

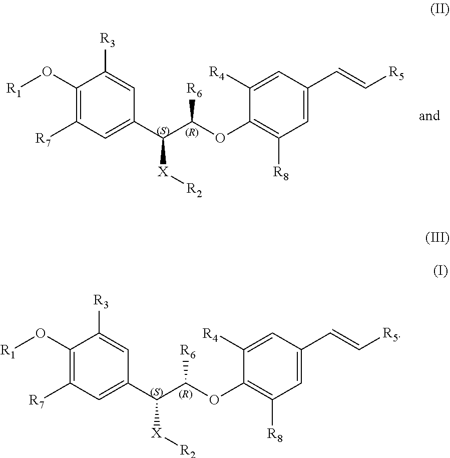

In some embodiments a threomer is provided selected from the following:

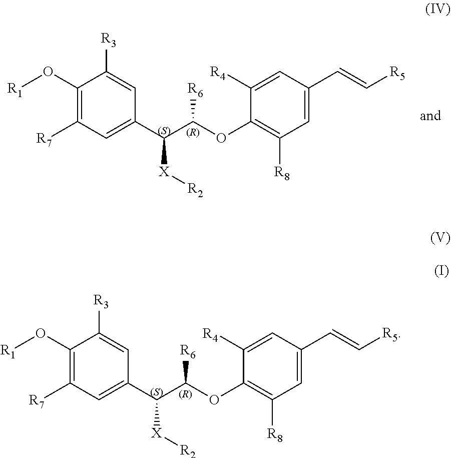

In some embodiments an erythromer is provided selected from the following:

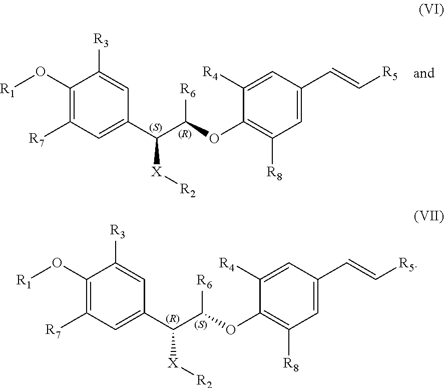

In some embodiments a threomer is provided selected from the following:

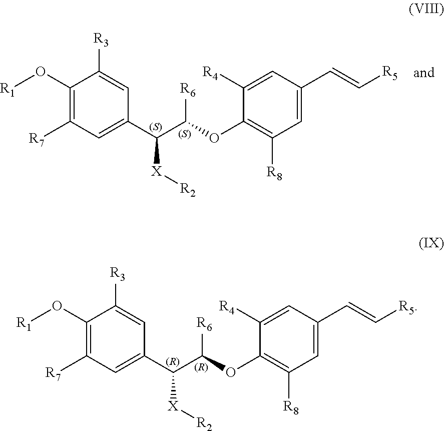

In some embodiments of the compounds of Formulae II-IX, X is O and R₂ is methyl, ethyl, propyl, butyl, isopropyl, isobutyl, or isopentyl. In some embodiments of the compounds of Formulae II-IX, X is NH and R₂ is H, methyl, ethyl, propyl, butyl, isopropyl, isobutyl, or isopentyl. In some embodiments of the compounds of Formulae II-IX, X is S and R₂ is H, methyl, ethyl, propyl, butyl, isopropyl, isobutyl, or isopentyl.

In some embodiments of the compounds of Formulae II-IX, R₁ is selected from the group consisting of H, methyl, and acetyl. In some embodiments of the compounds of Formulae II-IX, R₃ is H or methoxy. In some embodiments of the compounds of Formulae II-IX, R₄ is H or hydroxyl. In some embodiments of the compounds of Formulae II-IX, R₅ is selected from the group consisting of methyl, hydroxymethyl, methoxy, methoxymethyl. In some embodiments of the compounds of Formulae II-IX, R₆ is selected from the group consisting of methyl, hydroxyl, hydroxymethyl, and methoxymethyl. In some embodiments of the compounds of Formulae II-IX, R₇ is selected from the group consisting of H, hydroxyl, and methoxy. In some embodiments of the compounds of Formulae II-IX, R₈ is selected from the group consisting of H, hydroxyl, and methoxy.

In some embodiments of the compounds of Formulae II-IX, R₁ is H; R₃ is H or methoxy; R₄ is H or hydroxyl; R₅ is methyl, hydroxymethyl, methoxy, or methoxymethyl; R₆ is methyl; R₇ is methoxy; and R₈ is methoxy. In some embodiments of the compounds of Formulae II-IX, R₁ is H; R₃ is H; R₄ is H; R₅ is methyl; R₆ is methyl; R₇ is methoxy; R₈ is methoxy; and X is O.

In some embodiments an erythromer is provided selected from the following:

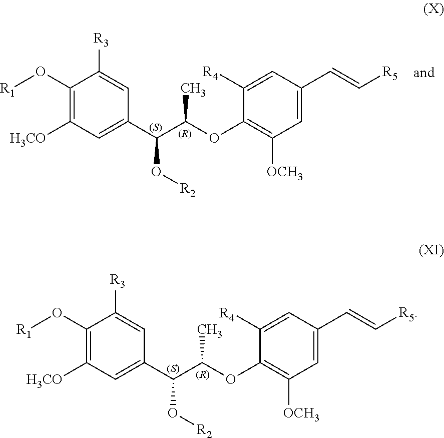

In some embodiments a threomer is provided selected from the following:

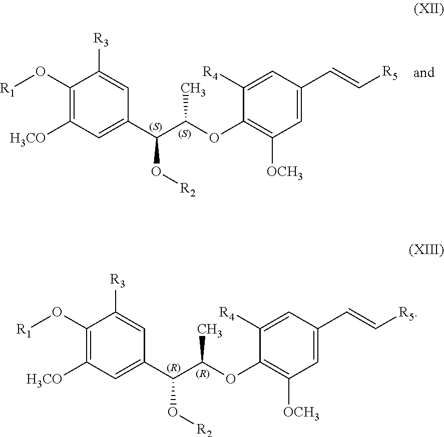

In some embodiments an erythromer is provided selected from the following:

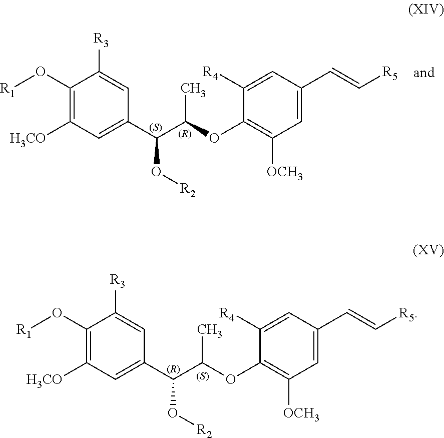

In some embodiments a threomer is provided selected from the following:

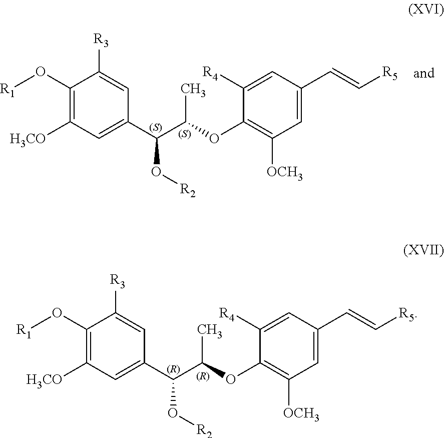

In some embodiments of the compounds of Formulae X-XVII, R₁ is selected from the group consisting of H, methyl, and acetyl. In some embodiments of the compounds of Formulae X-XVII, R₂ is methyl, ethyl, propyl, butyl, isopropyl, isobutyl, or isopentyl. In some embodiments of the compounds of Formulae X-XVII, R₃ is H or methoxy. In some embodiments of the compounds of Formulae X-XVII, R₄ is H or hydroxyl. In some embodiments of the compounds of Formulae X-XVII, R₅ is selected from the group consisting of methyl, hydroxymethyl, methoxy, methoxymethyl.

In some embodiments of the compounds of Formulae X-XVII, R₁ is H; R₂ is methyl, ethyl, propyl, butyl, isopropyl, isobutyl, or isopentyl; R₃ is H or methoxy; R₄ is H or hydroxyl; and R₅ is methyl, hydroxymethyl, methoxy, or methoxymethyl. In some embodiments of the compounds of Formulae X-XVII, R₁ is H; R₂ is methyl, ethyl, propyl, butyl, isopropyl, isobutyl, or isopentyl; R₃ is H; R₄ is H; and R₅ is methyl.

In some embodiments of the presently disclosed subject matter, the compound is selected from the following:

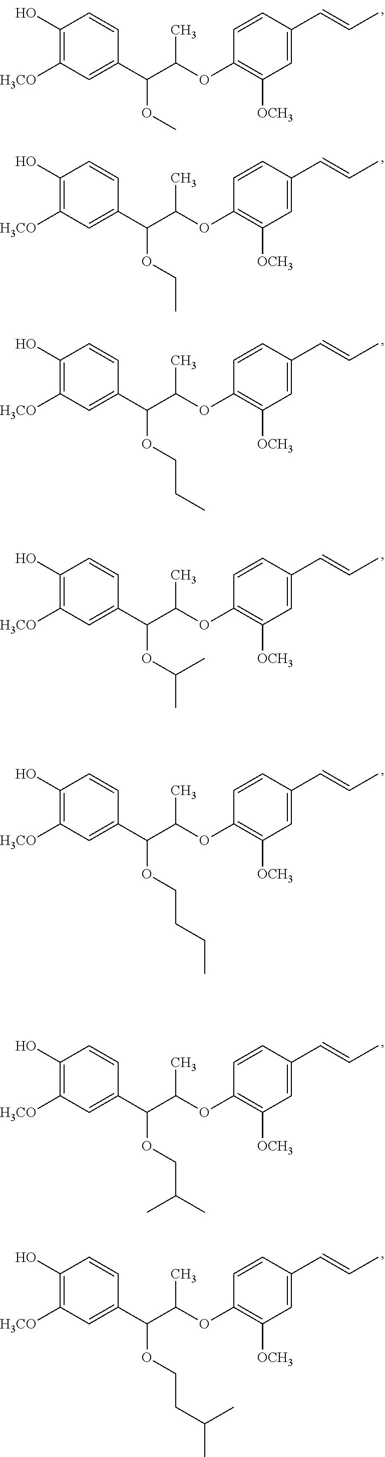

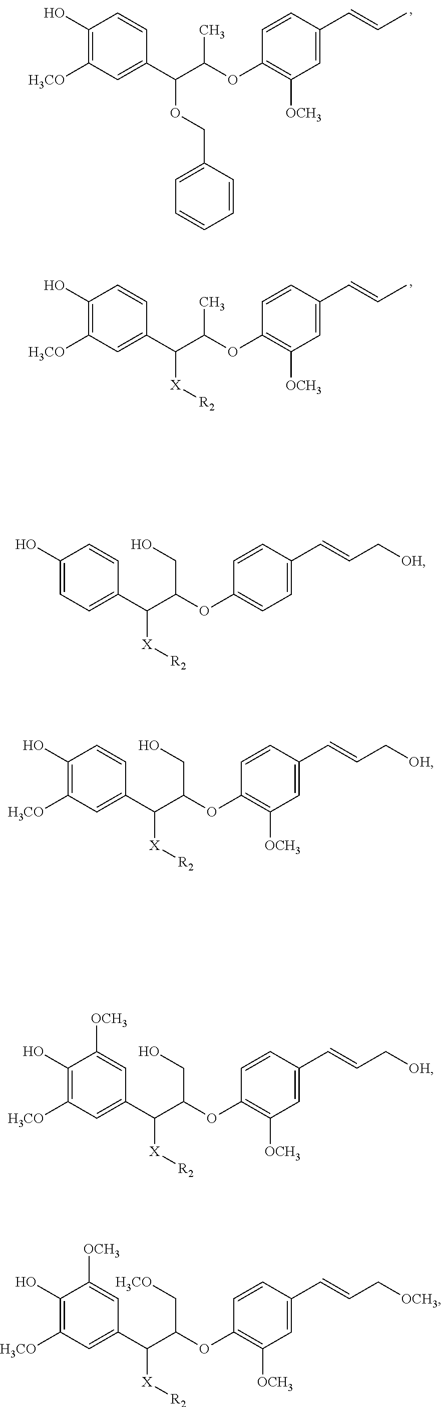

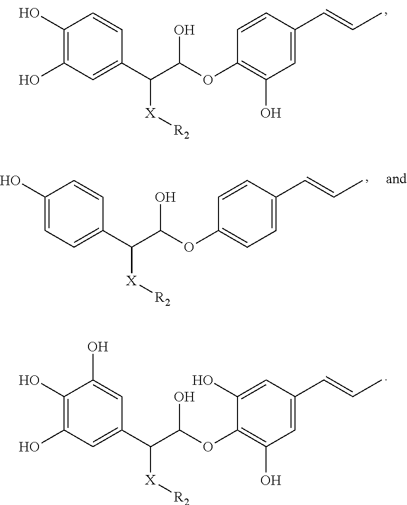

It is noted that in some instances compounds are depicted herein with regard to a particular stereochemistry, e.g., particular diastereomers, while in other instances, specific stereochemistry is not depicted. Unless otherwise specified, when a structure does not depict a specific stereochemistry, it is intended to be inclusive of the diastereomers as described herein or a mixture thereof.

The presently-disclosed subject matter further includes compositions comprising a compound as disclosed herein and a pharmaceutically-acceptable carrier.

The presently-disclosed subject matter further includes methods of using the compounds and compositions as disclosed herein. A method for conferring anti-cancer activity to a cancer cell, comprises contacting the cancer cell with an effective amount of a compound or composition as disclosed herein. In some embodiments, the conferring anti-cancer activity results in one or more of inhibiting proliferation of the cancer cell, inhibiting metastasis, and killing the cancer cell. In some embodiments, the cell is a cultured cell. In some embodiments, the cell is in a subject. In some embodiments, the subject is a mammal. In some embodiments, the cancer is a breast cancer. In some embodiments, the breast cancer is a basal-like tumor. In some embodiments, the breast cancer is triple negative breast cancer. In some embodiments, the triple negative breast cancer is an immunomodulatory (IM) subtype. In some embodiments, the method further comprises administering at least one additional component selected from the group consisting of: cytostatics, PARP inhibitors, and immune checkpoint inhibitors.

The presently-disclosed subject matter further includes methods of making the compounds as disclosed herein. An obstacle to developing lignan-derived bioactive agents is natural product extraction yield. Prior studies have relied on dismal yields of plant-extracted material to perform characterization and biological studies (Table 1). Although the compound shows great promise as a therapeutic agent, relying on natural sources is an untenable path forward. A synthetic method is essential for improving scalability.

TABLE 1
Summary of published natural sources of machilin
C and D and reported extraction yields.

| | | Bulk | | Machilin C/D | | | |
|---|---|---|---|---|---|---|---|
| Plant | | Material | | Extracted | | Extraction | |
| Species | Source | Mass | | Mass* | | % Yield | Reference |
| Machilus | Bark | 5 | kg | 14.1 | mg | 0.000282% | [18] |
| thunbergii | | | | | | | |
| Saururus | Roots | 2 | kg | 15 | mg | 0.000750% | [15] |
| chinensis | | | | | | | |
| Saururus | Roots | 1 | kg | 48 | mg | 0.004800% | [24] |
| chinensis | | | | | | | |
| Saururus | Leaves | 50 | g | 0.07 | mg | 0.00014% | [15] |
| chinensis | | | | | | | |
| Myristica | Aril | 24 | kg | 8.2 | mg | 0.0000341% | [38] |
| fragrans | | | | | | | |

*If both machilin C and D were isolated, their masses were combined for this table's purpose.

There is only one previously reported synthetic method towards the production of either machilin C or D via oxidative coupling for biomimicry or synthetic purposes. In 2010, Xia et. al. published a twelve-step asymmetric total synthesis of machilin C with 45-95% yield at each step.[39] While stereoselective, this method lacks efficiency and scalability, rendering it unsustainable for further development.

Disclosed herein is a convenient synthetic method allowing for the rapid production of machilin C and D and unique analogs thereof. This method uses another natural product, isoeugenol, as the feedstock. Isoeugenol is used worldwide as an essential oil in the manufacturing of laundry and cleaning products, perfumes, cosmetics, and foods.[40] Isoeugenol is an abundant feedstock and also can be easily synthesized from a bounty of other natural materials.[41, 42] It also has the potential to be produced from lignin, a ubiquitous biopolymer in need of valorization. [43] Utilization of isoeugenol positions this method as economical, scalable, and accessible to worldwide markets.

The dimerization of isoeugenol to synthesize another lignan, dehydrodiisoeugenol (licarin A), has been thoroughly reported and the mechanism is well understood (Scheme 1).^{[44-46]} However, none of these sources have recognized the secondary products obtained from this synthesis: machilin C&D.

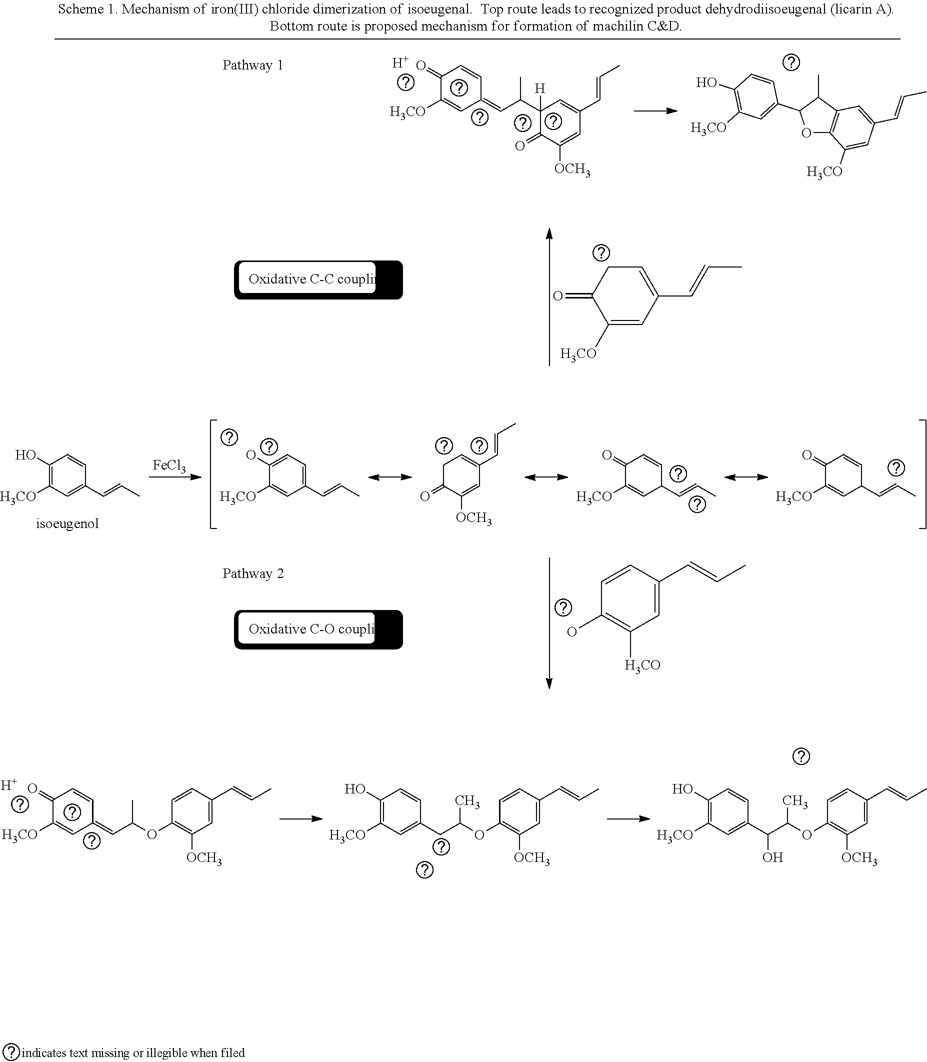

As disclosed herein, this synthetic method and penultimate cationic site have been leveraged to both recreate the natural products and develop unique compounds based on the dimer scaffold. One goal is to improve upon the cytotoxicity and anti-tumor properties previously reported while advancing an efficient synthetic method.

In some embodiments of the presently-disclosed subject matter, a method of making a compound as disclosed herein comprises dimerizing isoeugenol, an isoeugnol analog, or a monolignol in a reaction with a compound having the structure of R₂OH, wherein R₂ is selected from the group consisting of H, lower alkyl, benzyl, ethyl 2-hydroxy, cyclohexyl, methylcyclohexyl and N,N-dimethylaminoethyl. In some embodiments, the isoeugenol analog is selected from the group consisting of dehydroxzingerone, (E)-1,2-benzenediol, 4-(1-propenyl), 4-(1E)-1-propenylphenol, 5-(2-Propen-1-yl)-1,2,3-benzenetriol. In some embodiments, the monolignol is selected from the group consisting of p-coumaryl alcohol, coniferyl alcohol, and sinapyl alcohol.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, methylpropynyl, butynyl, pentynyl, hexynyl, heptynyl, and allenyl groups. “Branched” refers to an alkyl group in which a lower alkyl group, such as methyl, ethyl, or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

“Diastereomers” are stereoisomers that are not mirror images of each other and cannot be superimposed. Diastereomers have different spatial arrangements of atoms or groups around one or more chiral centers, leading to distinct physical and chemical properties.

In connection with stereoisomers, (S) and (R) designations come from the Cahn-Ingold-Prelog (CIP) priority rules and describe the absolute configuration around a chiral center. (R) is from the Latin term “rectus,” meaning “right,” and indicates that, when the groups attached to the chiral center are ranked by priority, the priority decreases in a clockwise direction when viewed from a particular angle. (S) is from the Latin word “sinister,” meaning “left,” and indicates that the priority of the attached groups decreases in a counterclockwise direction. The (R) or (S) configuration is determined by assigning priorities based on atomic number and analyzing the spatial arrangement of these groups.

The terms “Erythro” and “Threo” are used specifically to describe diastereomers of molecules with two chiral centers. (Erythro) refers to a pair of diastereomers where the substituents on the chiral centers are on the same side in a Fischer projection. When looking at the molecule, the groups of higher priority are on the same side of the main carbon chain. In contrast, the (Threo) designation is used for diastereomers where the substituents on the chiral centers are on opposite sides in a Fischer projection, in which the groups of higher priority are located on opposite sides of the main carbon chain.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Synthesis

Briefly, the natural products machilin C (threomer) and machilin D (erythromer) (1) were synthesized under mild conditions by combining aqueous iron (III) chloride with isoeugenol in acetone at room temperature (Scheme 2). Similarly, the novel analogs were synthesized using our established protocol for the synthesis of machilin C/D with the addition of methanol (2), ethanol (3), propanol (4), isopropanol (5), butanol (6), isobutanol (7), or isopentanol (8) (Scheme 2). Additional exemplary synthetic schemes are also provided (Scheme 3-9).

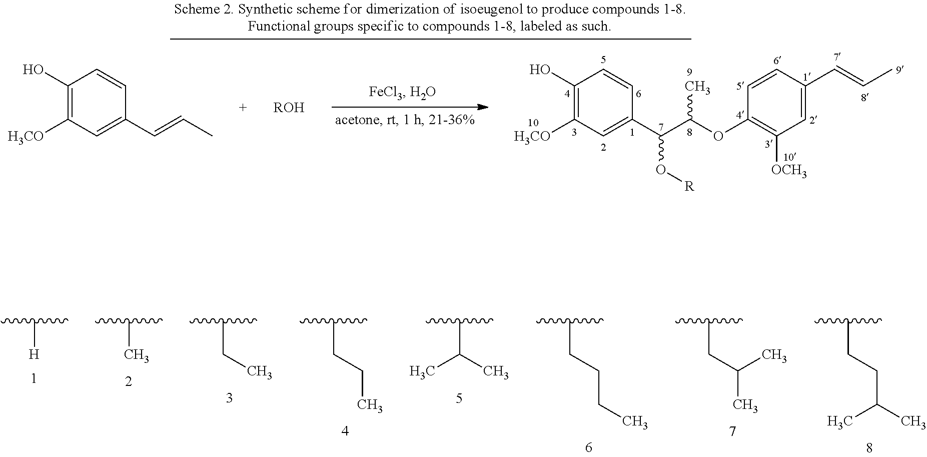

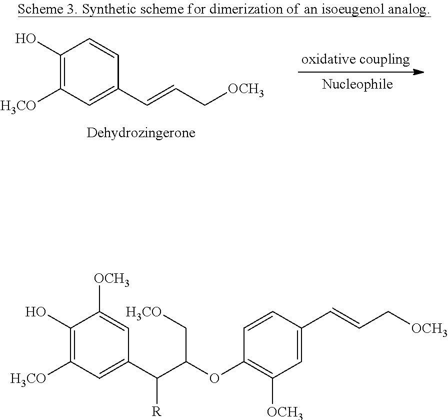

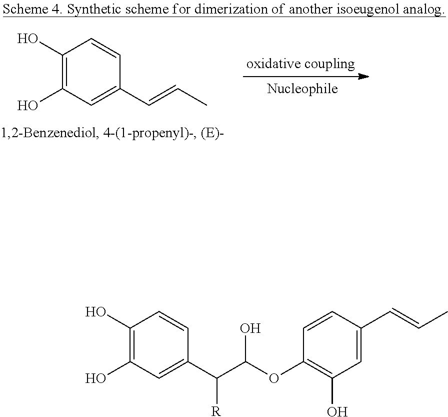

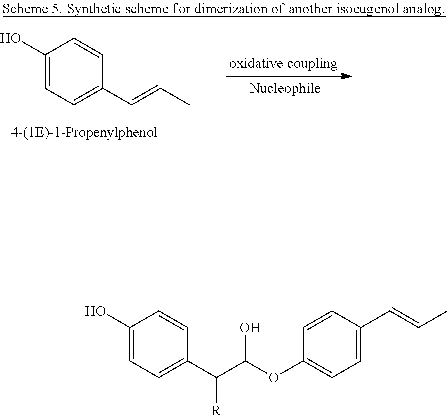

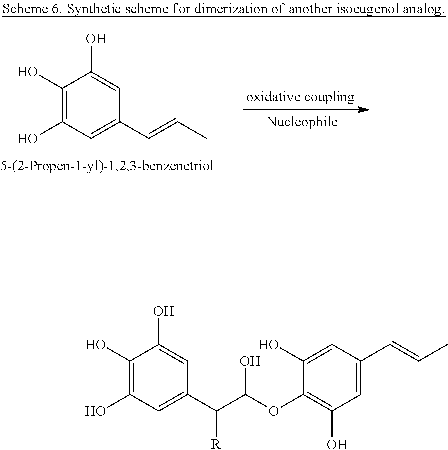

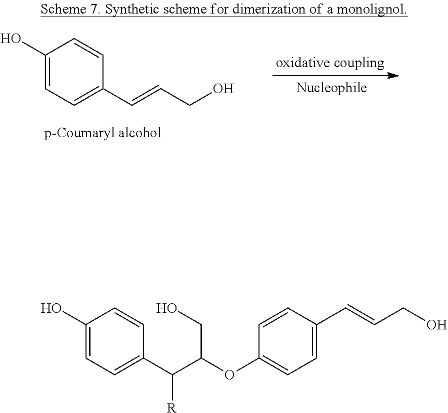

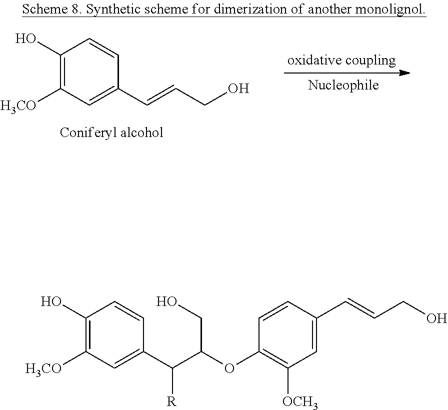

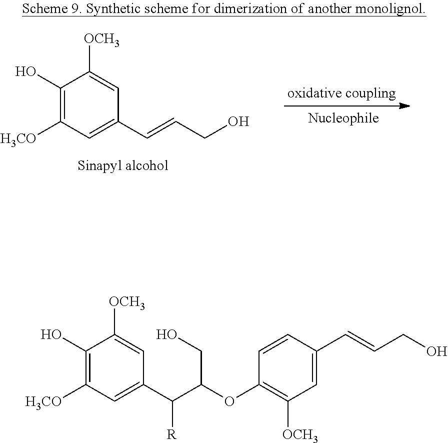

The R group presented in the forgoing Schemes 3-9 can be, for example, alcohol-based R-groups, alkyl amine R-groups, or thio R-groups, such as presented in Table 2.

TABLE 2
R-Groups
Alcohol Based R-Groups

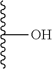 [SMILES: *O]

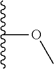 [SMILES: *OC]

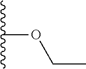 [SMILES: *OCC]

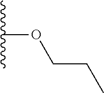 [SMILES: *OCCC]

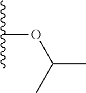 [SMILES: *OC(C)C]

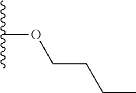 [SMILES: *OCCCC]

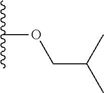 [SMILES: *OCC(C)C]

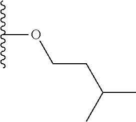 [SMILES: *OCCC(C)C]

Alkyl Amine R-Groups

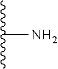 [SMILES: *N]

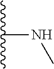 [SMILES: *NC]

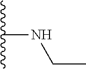 [SMILES: *NCC]

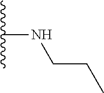 [SMILES: *NCCC]

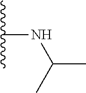 [SMILES: *NC(C)C]

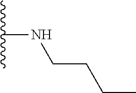 [SMILES: *NCCCC]

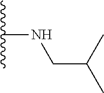 [SMILES: *NCC(C)C]

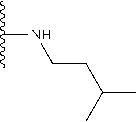 [SMILES: *NCCC(C)C]

Thio R-Groups

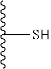 [SMILES: *S]

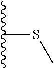 [SMILES: *SC]

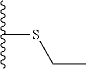 [SMILES: *SCC]

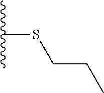 [SMILES: *SCCC]

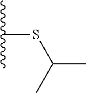 [SMILES: *SC(C)C]

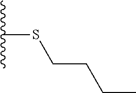 [SMILES: *SCCCC]

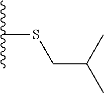 [SMILES: *SCC(C)C]

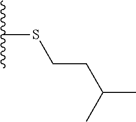 [SMILES: *SCCC(C)C]

Reactions were carried out under ambient conditions in air unless otherwise stated. Solvents were of ACS grade purchased from Greenfield Global. Isoeugenol was purchased from Alfa Aesar and Iron(III) chloride was purchased from Ward's Science. Deuterated solvents were purchased from Cambridge Isotope Laboratories (Andover, MA). All reagents were used as received. Dulbecco's Modified Eagle Medium (DMEM), phosphate buffered solution (PBS), trypsin, fetal bovine serum (FBS), penicillin, streptomycin, and amphotericin B were purchased from Corning, Inc. and used as received. Insulin was purchased from Sigma Aldrich and used as received. MammoCult Human Medium, heparin, and hydrocortisone were obtained from StemCell Technologies.

Isoeugenol (500 mg, 3.045 mmol) was placed in a 250 mL Erlenmeyer flask and 25 mL of acetone was added. While stirring, the appropriate reagent as described in Table 3 was added to the flask. In a separate 25 mL beaker, iron (III) chloride (463 mg, 2.85 mmol) was combined with 15 mL DI water and transferred to a 30 mL syringe. A syringe pump was used to add the solution into the Erlenmeyer flask at a rate of 15 mL/hour, with stirring at room temperature. At the end of one hour, the reaction mixture was transferred to a separatory funnel containing 50 mL DI water and 50 mL ethyl acetate. The mixture was extracted three times and all organic layers combined and rotary evaporated. The resulting product was a slightly yellow viscous oil. Isolation of the compound was achieved via chromatographic separation on silica gel using a 100 mL each of the following solvent mixes: 100% hexanes, 90% hexanes/ethyl acetate, 80% hexanes/ethyl acetate, 60% hexanes/ethyl acetate. The contents of the resulting fractions were identified via TLC and HPLC-MS. The purified fractions were combined and rotary evaporated to isolate a slightly yellow, viscous oil.

TABLE 3
Variable reagents used to synthesize compounds
1-8 as described in above preparation.

| Compound | Reagent | Volume (mL) | mols |
|---|---|---|---|
| 1 | Water (DI) | 25 | 1.4 |
| 2 | Methanol | 50 | 1.2 |
| 3 | Ethanol | 50 | 0.9 |
| 4 | Propanol | 50 | 0.7 |
| 5 | Butanol | 50 | 0.7 |
| 6 | Isopropanol | 50 | 0.5 |
| 7 | Isobutanol | 50 | 0.5 |
| 8 | Isopentanol | 50 | 0.5 |

### Example 2: Characterization

All products were obtained in a 21-36% product yield. Compounds 1-8 were characterized by ¹H and 13C spectroscopy, high resolution mass spectrometry, and the purity was confirmed by HPLC (Spectra and chromatograms not shown).

The synthetic method is not stereoselective and produces a mix of diastereomers. For preliminary cell viability assays, we proceeded with the diastereomeric mixture to evaluate the cytotoxic effect of the compounds on cells. Therefore, each HPLC result exhibits two peaks indicative of the diasteromeric mixtures, and the NMR spectra display dual peaks for protons in range of the two chiral centers, which has been well annotated where appropriate in the supplementary information. Machilin C&D as well as similar lignans naturally exist as a diastereomeric pair in varying ratios.^{[47-49]} Therefore, the two compounds were investigated first as a pair and then as separate entities.

¹H and ¹³C NMR: NMR spectra were recorded on 500 MHz JEOL ECZr spectrometer. NMR experiments were recorded in CDCl₃ 25° C. and calibrated with reference points of ¹H NMR (CDCl₃ δ=7.26 ppm) and ¹³C NMR (CDCl₃ δ=77.16).

HRMS Method: High-resolution mass spectra (HRMS) were recorded on a Thermo Q Exactive Hybrid Quadrupole-Orbitrap mass spectrometer. Compounds 1-8 were prepared at a concentration of 1 mg/mL in 50% acetonitrile/DI water. Solution was directly infused at a rate of 20 μL/min and all spectra were collected for 1 minute in positive ion mode.

HPLC Method: Liquid chromatograms were obtained with RP-HPLC using an Agilent Technologies 1100 series HPLC instrument with an Agilent Eclipse Plus C18 column (4.6 mm×100 mm; 3.5 μm particle size). Compounds 1-8 were prepared at a concentration of 1 mg/mL in 50% acetonitrile/DI water. Injection volume was set to 10 μL. Flow rate was set to 1 mL/min with a linear elution gradient of 0.1% trifluoroacetic acid in water/0.05% formic acid in acetonitrile. Gradient was held at 50:50 for 2 minutes then ramped to 0:100 by 10 minutes. Total run time was 15 minutes. Multiple wavelength detection was used with data collected at 210, 220, 230, 260, and 280 nm. All compounds were found to be >95% pure.

Alternate LC Method: Liquid chromatograms were obtained with RP-HPLC using Shimadzu LC-36AD UPLC instrument with a Kromasil ExternityXT C18 column (2.1 mm×50 mm; 1.8 μm particle size) coupled to a Q Exactive Orbitrap mass spectrometer. Compounds 7A & 7B were prepared at a concentration of 10 μg/mL in 50% acetonitrile/DI water. Injection volume was set to 5 μL. Flow rate was set to 0.35 L/min with a linear elution gradient of 0.1% formic acid in water/0.1% formic acid in acetonitrile. Gradient was held at 50:50 for 2 minutes then ramped up to 0:100 by 8 minutes. Total run time was 10 minutes. TIC was used as method of detection.

Compound 1: 36% yield, colorless oil, ¹H-NMR (500 MHz; CDCl₃): δ 6.98-6.95 (m, 1H), 6.92 (s, 1H), 6.91 (d, J=1.7 Hz, 1H), 6.87 (q, J=2.3 Hz, 2H), 6.85 (d, J=2.2 Hz, 1H), 6.35 (dd, J=15.7, 1.7 Hz, 1H), 6.15 (dq, J=15.7, 6.6 Hz, 1H), 5.60 (s, 1H), 4.61 (d, J=8.4 Hz, 1H), 4.08 (dq, J=8.4, 6.3 Hz, 1H), 3.92 (s, 3H), 3.89 (s, 3H), 1.88 (dd, J=6.6, 1.6 Hz, 3H), 1.16 (d, J=6.2 Hz, 3H). ¹³C-NMR (126 MHz; CDCl₃): δ 150.9, 146.84, 146.66, 145.6, 132.0, 130.5, 125.0, 120.8, 119.1, 118.9, 114.2, 109.44, 109.25, 84.4, 78.6, 56.02, 55.83, 18.5, 17.1 ESI⁺ HRMS: m/z 367.15039 [M+Na](calcd. for C₂₀H₂₄O₅Na, 367.15215)

Compound 2: 22% yield, colorless oil, ¹H-NMR (500 MHz; CDCl₃): δ 6.91 (d, J=1.9 Hz, 1H), 6.89 (s, 1H), 6.88 (d, J=3.0 Hz, 2H), 6.82 (dt, J=8.1, 1.7 Hz, 2H), 6.33 (dd, J=15.7, 1.5 Hz, 1H), 6.10 (dq, J=15.7, 6.6 Hz, 1H), 5.62 (s, 1H), 4.45 (quintet, J=6.4 Hz, 1H), 4.28 (d, J=6.3 Hz, 1H), 3.89 (s, 3H), 3.84 (s, 3H), 3.28 (s, 3H), 1.86 (dd, J=6.6, 1.6 Hz, 3H), 1.07 (d, J=6.4 Hz, 3H). ¹³C-NMR (126 MHz; CDCl₃): δ 151.8, 150.7, 147.2, 146.7, 145.5, 132.1, 130.79, 130.72, 124.1, 121.2, 118.8, 116.9, 114.0, 109.9, 109.6, 86.7, 79.2, 57.2, 56.06, 56.02, 18.5, 16.6 ESI⁺ HRMS: m/z 381.16574 [M+Na](caled. for C21H₂₆O₅Na, 381.16780)

Compound 3: 28% yield, colorless oil, ¹H-NMR (500 MHz; CDCl₃): δ 6.93 (s, 1H), 6.84 (s, 2H), 6.83 (d, J=2.0 Hz, 1H), 6.76 (dd, J=8.2, 2.0 Hz, 1H), 6.68 (d, J=8.2 Hz, 1H), 6.30 (dd, J=15.7, 1.6 Hz, 1H), 6.08 (dq, J=15.7, 6.6 Hz, 1H), 5.55 (s, 1H), 4.36 (t, J=4.6 Hz, 1H), 4.33 (d, J=5.6 Hz, 1H), 3.85 (s, 3H), 3.78 (s, 3H), 3.52-3.40 (m, 2H), 1.85 (dd, J=6.6, 1.6 Hz, 3H), 1.35 (d, J=6.0 Hz, 3H), 1.20 (t, J=7.0 Hz, 3H). ¹³C-NMR (126 MHz; CDCl₃): δ 150.9, 146.9, 146.6, 145.2, 132.28, 132.10, 130.8, 124.3, 120.8, 118.9, 117.4, 114.0, 110.1, 109.9, 83.7, 79.9, 65.0, 56.16, 56.14, 18.6, 15.9, 15.6. ESI⁺ HRMS: m/z 395.18173 [M+Na](caled. for C₂₂H₂₈O₅Na, 395.18344)

Compound 4: 24% yield, colorless oil, ¹H-NMR (500 MHz; CDCl₃): δ 6.92 (s, 1H), 6.83 (s, 2H), 6.82 (d, J=1.9 Hz, 1H), 6.75 (dd, J=8.2, 2.0 Hz, 1H), 6.66 (d, J=8.2 Hz, 1H), 6.28 (dd, J=15.7, 1.5 Hz, 1H), 6.07 (dq, J=15.7, 6.6 Hz, 1H), 5.52 (s, 1H), 4.33 (dd, J=5.3, 1.6 Hz, 1H), 4.32 (s, 1H), 3.84 (s, 3H), 3.77 (s, 3H), 3.39-3.29 (m, 2H), 1.83 (dd, J=6.6, 1.6 Hz, 3H), 1.60 (t, J=7.1 Hz, 2H), 1.35 (d, J=5.9 Hz, 3H), 0.91 (t, J=7.4 Hz, 3H). ¹³C-NMR (CDCl₃): δ 150.7, 146.7, 146.4, 145.0, 132.05, 131.98, 130.7, 124.1, 120.6, 118.7, 117.1, 113.8, 110.0, 109.8, 83.6, 79.8, 71.2, 55.99, 55.94, 23.1, 18.5, 15.8, 10.8 ESI⁺ HRMS: m/z 409.19751 [M+Na](caled. for C₂₃H₃₀O₅Na, 409.19910)

Compound 5: 21% yield, colorless oil, ¹H-NMR (500 MHz; CDCl₃): δ 6.95 (d, J=1.7 Hz, 1H), 6.87 (d, J=1.7 Hz, 1H), 6.84 (s, 1H), 6.82 (d, J=1.9 Hz, 1H), 6.75 (dd, J=8.2, 2.0 Hz, 1H), 6.63 (d, J=8.3 Hz, 1H), 6.29 (dd, J=15.7, 1.6 Hz, 1H), 6.07 (dq, J=15.7, 6.6 Hz, 1H), 5.52 (s, 1H), 4.45 (d, J=5.9 Hz, 1H), 4.30 (quintet, J=6.1 Hz, 1H), 3.85 (s, 3H), 3.78 (s, 3H), 3.58 (dt, J=12.2, 6.1 Hz, 1H), 1.84 (dd, J=6.6, 1.6 Hz, 3H), 1.37 (d, J=6.2 Hz, 3H), 1.17 (d, J=6.0 Hz, 3H), 1.11 (d, J=6.2 Hz, 3H). ¹³C-NMR (126 MHz; CDCl₃): δ 150.6, 146.9, 146.4, 145.0, 132.9, 131.8, 130.7, 124.0, 120.6, 118.7, 116.5, 113.8, 110.1, 109.7, 81.0, 79.7, 69.7, 56.03, 56.00, 23.5, 21.4, 18.5, 16.1 ESI⁺ HRMS: m/z 409.19766 [M+Na](caled. for C₂₃H₃₀O₅Na, 409.19910)

Compound 6: 23% yield, colorless oil, ¹H-NMR (500 MHz; CDCl₃): δ 6.93 (s, 1H), 6.84 (d, J=1.5 Hz, 2H), 6.83 (d, J=2.0 Hz, 1H), 6.76 (dd, J=8.3, 2.0 Hz, 1H), 6.68 (d, J=8.3 Hz, 1H), 6.30 (dd, J=15.7, 1.6 Hz, 1H), 6.08 (dq, J=15.7, 6.6 Hz, 1H), 5.56 (s, 1H), 4.34 (t, J=4.1 Hz, 1H), 4.33 (d, J=3.7 Hz, 1H), 3.85 (s, 3H), 3.78 (s, 3H), 3.44-3.33 (m, 2H), 1.85 (dd, J=6.6, 1.6 Hz, 3H), 1.57 (dqd, J=8.6, 6.5, 4.4 Hz, 2H), 1.42-1.37 (m, 2H), 1.36-1.35 (m, 3H), 0.90 (t, J=7.4 Hz, 3H). ¹³C NMR (126 MHz; CHLOROFORM-D): δ 150.7, 146.7, 146.4, 145.0, 132.05, 132.00, 130.7, 124.1, 120.6, 118.7, 117.1, 113.8, 110.0, 109.8, 83.7, 79.8, 69.2, 55.99, 55.94, 32.0, 19.5, 18.5, 15.8, 14.0 ESI⁺ HRMS: m/z 423.21307 [M+Na](caled. for C₂₄H₃₂O₅Na, 423.21475)

Compound 7: 21% yield, colorless oil, ¹H-NMR (500 MHz; CDCl₃): δ 6.93 (s, 1H), 6.84 (d, J=2.3 Hz, 2H), 6.83 (d, J=2.0 Hz, 1H), 6.76 (dd, J=8.3, 2.0 Hz, 1H), 6.68 (d, J=8.3 Hz, 1H), 6.30 (dd, J=15.8, 1.6 Hz, 1H), 6.08 (dq, J=15.7, 6.6 Hz, 1H), 5.56 (s, 1H), 4.36 (dd, J=9.4, 6.0 Hz, 1H), 4.32 (t, J=5.9 Hz, 1H), 3.85 (s, 3H), 3.78 (s, 3H), 3.19-3.11 (m, 2H), 1.93-1.87 (m, 1H), 1.85 (dd, J=6.6, 1.6 Hz, 3H), 1.37 (d, J=5.9 Hz, 3H), 0.92 (t, J=6.4 Hz, 6H). ¹³C-NMR (126 MHz; CHLOROFORM-D): δ 150.8, 146.8, 146.4, 145.0, 132.1, 130.7, 124.1, 120.7, 118.7, 117.1, 113.9, 110.05, 109.86, 83.9, 80.0, 76.4, 56.06, 55.97, 28.8, 19.65, 19.56, 18.5, 15.9. ESI⁺ HRMS: m/z 423.21323 [M+Na](caled. for C₂₄H₃₂O₅Na, 423.21475)

Compound 8: 24% yield, colorless oil, ¹H-NMR (500 MHz; CDCl₃): δ 6.93 (s, 1H), 6.84 (s, 2H), 6.84 (s, 1H), 6.76 (dd, J=8.3, 1.9 Hz, 1H), 6.67 (d, J=8.3 Hz, 1H), 6.30 (dd, J=15.9, 1.6 Hz, 1H), 6.11 (td, J=10.3, 5.4 Hz, 1H), 5.55 (s, 1H), 4.36 (t, J=6.3 Hz, 1H), 4.33 (s, 1H), 3.85 (s, 3H), 3.78 (s, 3H), 3.43 (ddd, J=10.1, 7.4, 3.4 Hz, 2H), 1.85 (d, J=1.4 Hz, 3H), 1.74 (dt, J=13.5, 6.7 Hz, 1H), 1.48 (qd, J=6.7, 2.5 Hz, 2H), 1.35 (d, J=5.9 Hz, 3H), 0.89 (d, J=6.7 Hz, 6H). ¹³C-NMR (126 MHz; CDCl₃): δ 150.8, 146.8, 146.4, 145.0, 132.1, 130.81, 130.71, 124.1, 120.7, 118.7, 117.1, 113.9, 110.0, 109.8, 83.8, 79.9, 67.9, 56.02, 55.98, 38.9, 25.1, 22.7, 18.5, 15.9. ESI⁺ HRMS: m/z 437.22849 [M+Na](caled. for C25H₃₄O₅Na, 437.23039)

### Example 3: Cell Line Maintenance

All cell lines were purchased from ATCC and kept in a humidified incubator at 37° C. with 5-10% CO₂. MDA-MB-231 and MDA-MB-468 were grown in DMEM supplemented with 10% FBS, 1% amphotericin and 1% penicillin/streptomycin. SUM159 cells were grown in DMEM supplemented with 10% FBS, 1% amphotericin, 1% penicillin/streptomycin, and 5 μg/mL insulin. DMEM, FBS, amphotericin, and penicillin/streptomycin were purchased from Corning Inc. and used as purchased. Insulin was purchased from Sigma Aldrich.

### Example 4: Cell Viability Assay

Cell Viability Assay for Compounds 1-8: The cytotoxicity for compounds 1-8 was assessed in three Triple Negative Breast Cancer cell lines: MDA-MB-231, MDA-MB-468, an SUM159. Cells were grown to 80% confluency in T75 flasks. Media was discarded and trypsin added for 5 minutes to detach cells. Media was added to neutralize the trypsin and mixture was transferred to centrifuge tube. After centrifugation at 2000 RPM for 5 minutes, the pellet was washed with PBS and resuspended in 2 mL appropriate media. Cell density was assessed via hemocytometer and solution diluted to 40,000 cells/mL. A 96-well clear bottom plate was prepared by adding media to rows 1 and 8 and columns 1, 2, 11, and 12 to prevent evaporation from impacting sample wells. Cells were plated in the inner wells with 100 μL per well (4,000 cells/well). Plates were placed in the incubator at 37° C. and 5-10% CO₂ overnight to allow cells to acclimate and adhere.

Compounds 1-8 were prepared as 80 mM stock solutions in DMSO. The stock was diluted 20 μL to 4 mL with the appropriate media to yield a new stock concentration of 400 μM and DMSO concentration of 0.5% v/v. This stock was used for six serial dilutions (1/2×) to obtain a range of concentrations from 6.25 to 400 μM. A control solution was prepared by diluting 20 μL DMSO in 4 mL media appropriate media. Cell plates were removed from the incubator and media removed from test wells. Plain media was left in rows 1 and 8 and columns 1, 2, 11, and 12. The control solution was applied to test wells in column 3 and then compound solutions applied from lowest to highest concentration in columns 4-10 (seven total concentrations). Plates were placed in the incubator at 37° C. and 5-10% CO₂ for 24 hours.

Plates were removed from the incubator and treatment solutions removed. A 0.5 mg/mL solution of MTT (3-(4,5-dimethylthiazol-2-yl)-2,5-diphenyl-2H-tetrazolium bromide) in appropriate media was added to each well (100 μL) and plates were placed in incubator at 37° C. and 5-10% CO₂ for 3 hours. At the endpoint, the MTT solution was removed and replaced with 100 μL DMSO. Plates were then read on a Biotek Synergy H1 Plate Reader at 570 nm (peak absorbance).

Cell Viability Assay for Compounds 7A and 7B: Protocol is same as that of Compounds 1-8, but concentrations tested were 12.5-100 μM (MDA-MB-468) and 6.25-75 μM (MDA-MB-231 and SUM159).

Statistical Analysis: For MTT assay, treated well data was normalized to the average control value and the resulting data was imported to GraphPad Prism. The six replicate data points for each concentration were used to create six dose-response curves using a nonlinear regression analysis. The IC₅₀ reported for each analysis is an average of the replicates with the SEM reported as error.

### Example 5: Inhibition of Triple Negative Breast Cancer (TNBC) Growth

To better understand the activity of the compounds with modifications relative to machilin C/D, as disclosed herein, the compounds were evaluated in TNBC cells, including MDA-MB-231 and MDA-MB-468 cells, using MTT viability assay and clonogenic assays.

Results indicate that the unique structures of the compounds disclosed herein improve TNBC inhibition and modulate distinct targets/pathways. For example, mechanism of action studies identified compound 7, an isobutoxy analog, to deplete c-MYC protein in a dose-dependent manner.

Mechanism and therapeutic impact of the derivatives disclosed herein in TNBC organoids and mouse models were studied. Cell lines were selected as being representative of TNBC cancer subtypes, including mesenchymal stem-like (MDA-MB-231), basal like-1 (MDA-MB-468) with different metabolic characteristics. Dose-response curves are fit to the data (four-parameter log-logistic model), and IC₅₀ values were calculated with Graphpad Prizm.

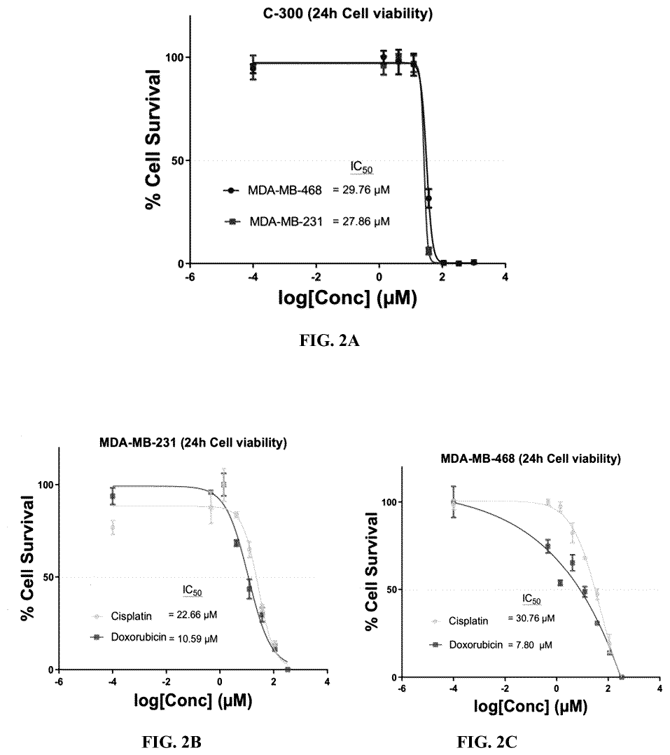

The results set forth in FIG. 2A-2C demonstrates that the structural modifications disclosed herein improve cytotoxic potential. For example, compound 7 exhibits activity across the cell lines in low μM concentrations compared to machilin D (˜100 μM, 24 h). Additionally, the IC₅₀ of C-300 at 24 h was comparable to FDA approved doxorubicin and cisplatin often prescribed to a subset of TNBC patients.

### Example 6: Inhibition of Colony Formation and c-MYC

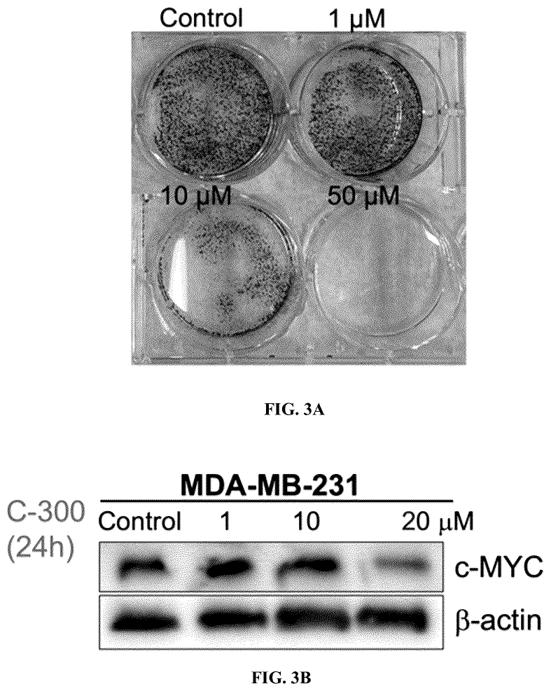

Colony formation is a unique phenotype of tumors, and their inhibition is important for determining potency. C-300 inhibits the growth of MDA-MB-231 colonies even at 1 mM in a 7-day clonogenic assay. To determine whether C-300 inhibits the stem cell marker genes, the protein level of c-MYC (a master regulator) were examined. C-300 was found to inhibit c-MYC protein in a dose-dependent manner FIG. 3A-3B.

### Example 7: Toxicity

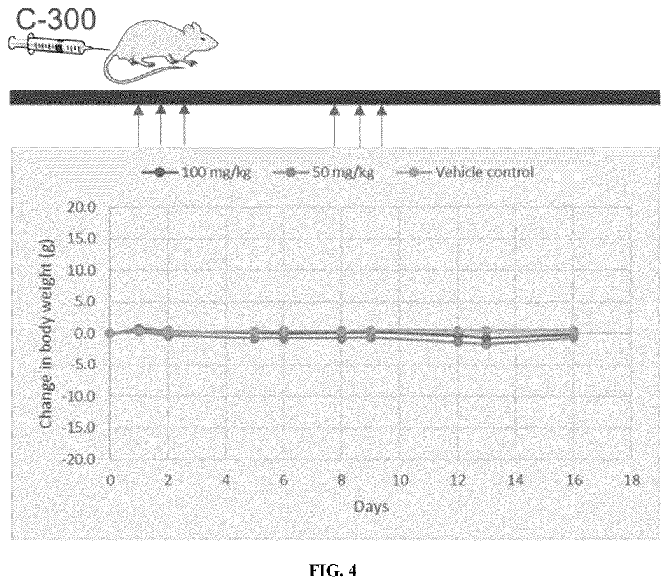

The potential toxicity of compound 7 was evaluated using 9-week-old C₅₇BL/6 female mice. The mice were injected (i.p.) with vehicle or 50 mg/kg, 100 mg/kg of compound 7 (3× a week for 2-weeks, 3 mice/group). The body weight was measured daily for 14 days. The liver, spleen, heart, lung, and kidney were collected for macroscopic examination. The results showed that control mice and mice treated with compound 7 at doses tested exhibited similar body weight gain (FIG. 4). Macroscopic examination did not reveal any signs of toxicity and the survival rate was 100% (data not shown).

### Example 8: Cell Viability, Compounds 1-8

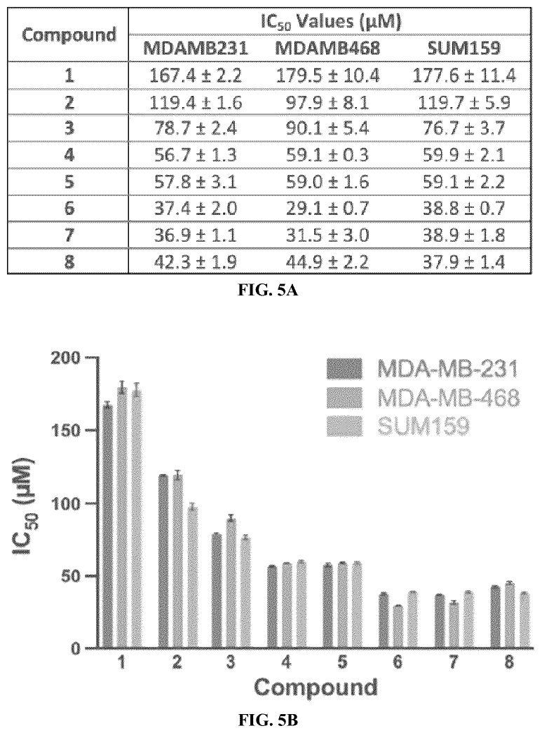

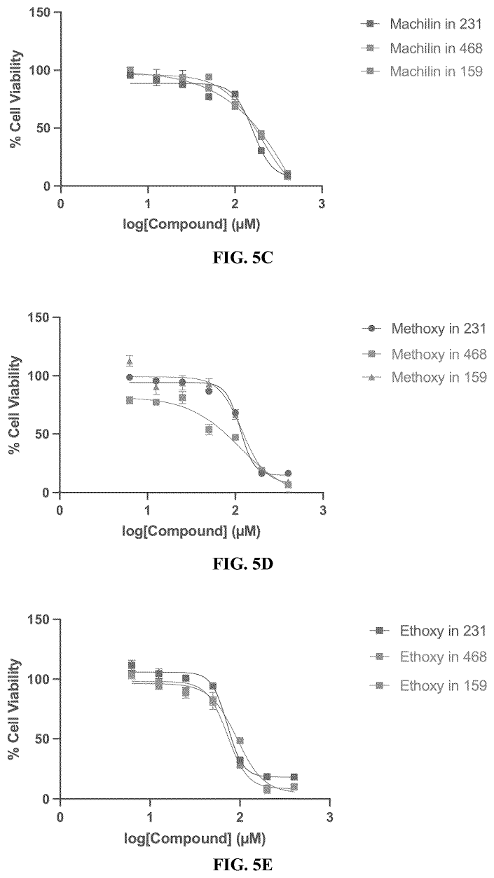

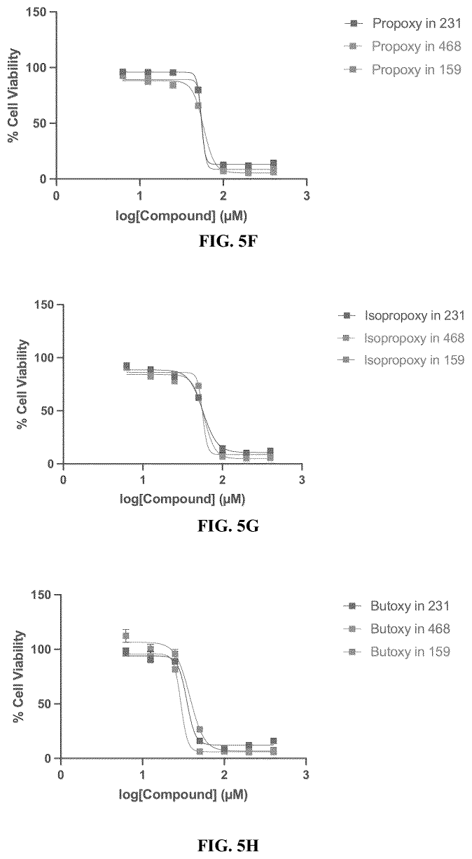

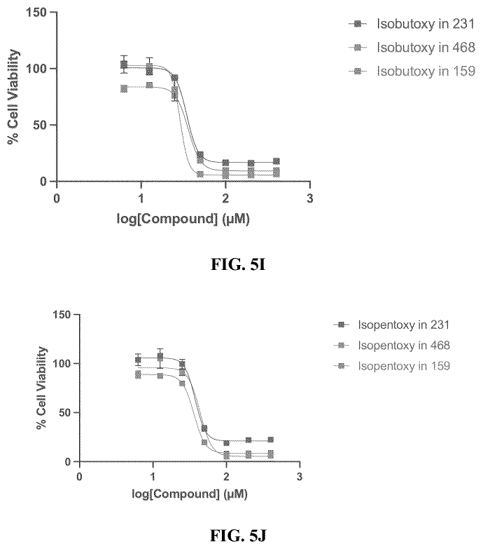

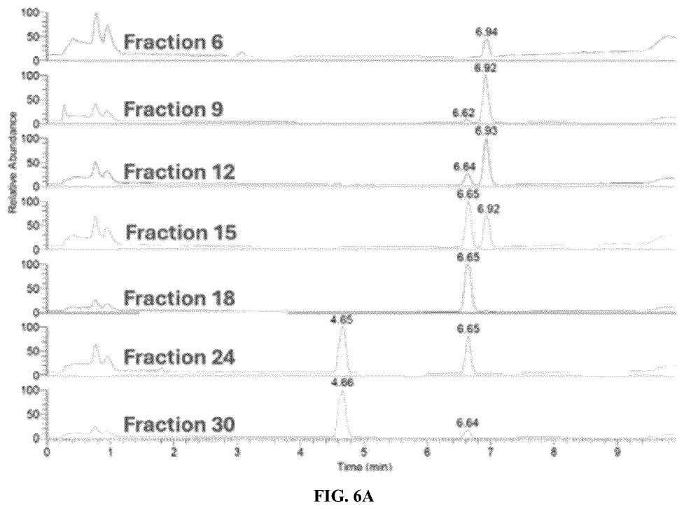

Initial structure activity relationship study to assess the effect of modification at the 7-0 position on biological function began with the evaluation of cell proliferation via the colorimetric MTT (3-(4,5-dimethylthiazol-2-yl)-2,5-diphenyltetrazolium bromide (MTT)) assay in three TNBC cell lines: MDA-MB-468, MDA-MB-231, and SUM159 following a 24 h exposure to compounds 1-8 (FIG. 5A). Machilin C and D exhibited IC₅₀ values of 167.4-179.5 μM, comparable to that reported by Zheng et. al.^{[14]} With extension of the alkyl chain at the 7-0 position, IC₅₀ values continue to decrease (FIG. 5B) and reach a plateau with compounds 6-8. Dose response curves for compounds 1-8 are provided in FIG. 5C-5J. It is contemplated that this structure-activity relationship (SAR) could be due to increased lipophilicity as the carbon chain lengthens. Higher lipophilicity values typically improve cell membrane penetration, allowing for better absorption.^{[50]} It is also feasible that this modification aids in binding to the target protein(s), a theory that can be further investigated after confirmation of targets.

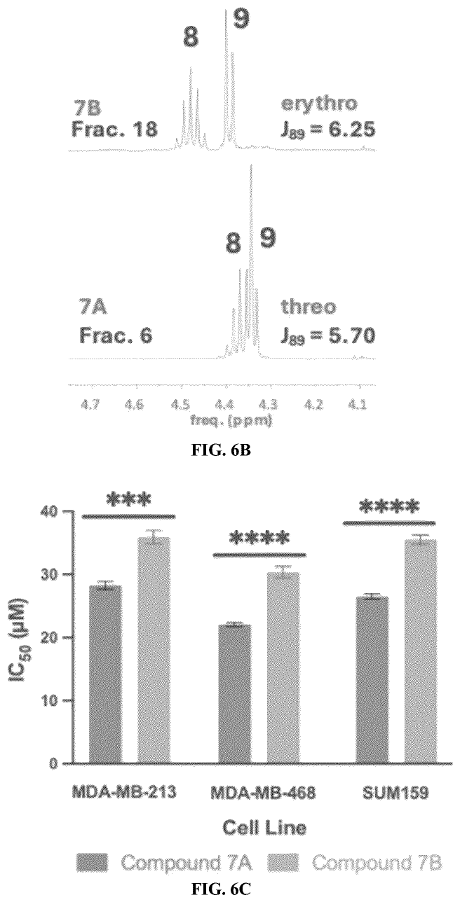

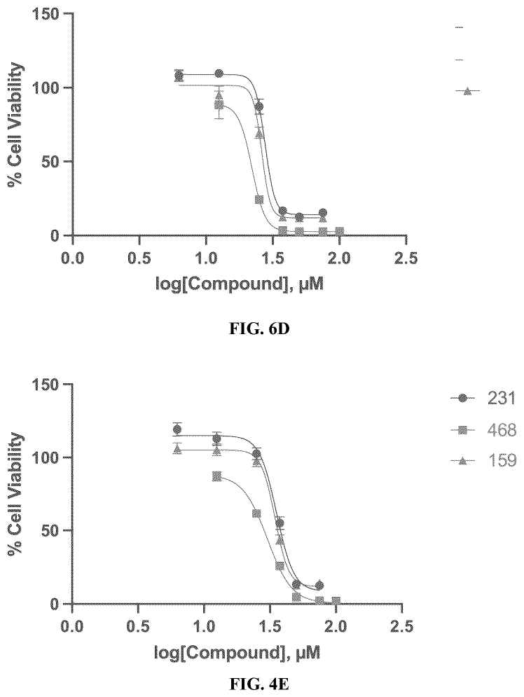

### Example 9: Impact of Stereochemical Configuration

Whereas previous studies have investigated the threo- and erythro-versions of this dimer scaffold (Machilin C and D respectively), none have performed direct comparison studies to investigate how the stereochemical configuration impacts biological activity. Compound 7 was chosen for this investigation due to its relatively low IC₅₀ value. The diastereomers were separated by manual silica column and each fraction was identified by HPLC spectroscopy (FIG. 6A). The first diastereomer to elute in normal phase chromatography is diastereomer A (7A) and the second is diastereomer B (7B). Note that elution order is reversed in LC chromatograms as a reverse-phase column was used. Fractions 6 and 18 were chosen as pure representatives of each diastereomer and their stereochemical configurations were assigned using ^{1H} NMR spectroscopy and LC chromatography. Previous research has indicated that lignan threomers exhibit smaller J-coupling values and exhibit upfield shifts when compared to erythromers. Both of these observations support 7A as the threomer and 7B as the erythromer when examining ¹H spectroscopy.^{[51]} In addition, a 2021 study by Asare et. el. found that in similar B—O-4′ lignan dimers, the erythromer typically elutes prior to the threomer in reverse-phase chromatography.^{[52]} The chromatographic results (FIG. 6B) indicate the 7B elutes prior to 7A in a reverse-phase system, confirming 7B as the erythromer and 7A as the threomer.

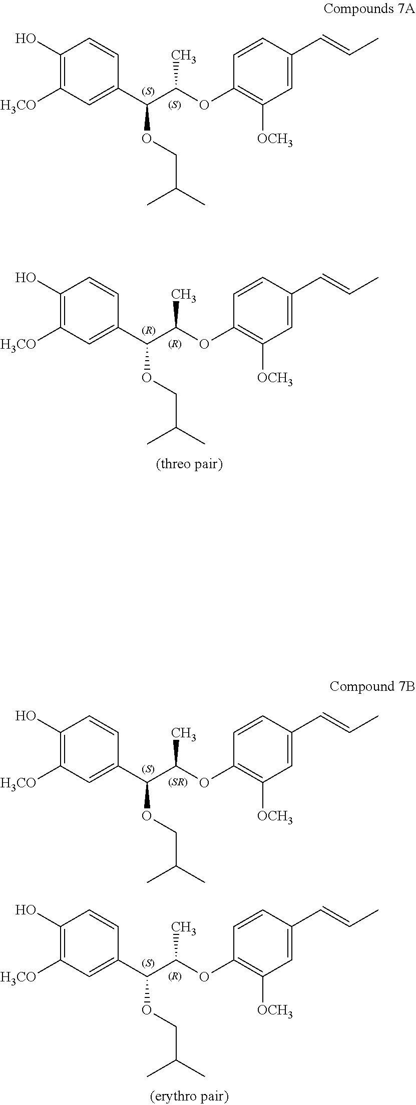

The impact of configuration on cytotoxicity was examined using MTT assay in three TNBC cell lines (FIG. 6C). It was found that the threomer (compound 7A) has significantly improved cytotoxicity values over 7B in all three cell lines. Dose response curves for compounds 1-8 are provided in FIG. 6D-6E.

### Example 10: Mammosphere Formation Assay

The ability of Compounds 1, 7A, and 7B to prevent mammosphere formation was assessed in SUM159, a Triple Negative Breast Cancer cell line. Cells were grown to 80% confluency in T75 flasks in DMEM supplemented with 10% FBS, 1% amphotericin, 1% penicillin/streptomycin, and 5 μg/mL insulin, Media was discarded and trypsin added for 5 minutes to detach cells. Media was added to neutralize the trypsin and mixture was transferred to centrifuge tube. After centrifugation at 2000 RPM for 5 minutes, the pellet was washed with PBS and resuspended in 2 mL mammosphere media. Mammosphere media consists of MammoCult Human Medium (StemCell Technologies) supplemented with Heparin (4 pig/ml, StermCellTechnologies) and Hydrocortisone (0.48 μg/ml, StemCell Technologies). Cell density was assessed via hemocytometer and solution diluted to 8,000 cells/mL in mammosphere media. A 24-well ultra-low-attachment clear bottom plate was prepared. Cells were plated in all wells with 500 μL per well (4,000 cells/well). Treatment conditions included control (0.5% DMSO), and compounds 1, 7A, and 7B at both half and full IC₅₀ concentrations. All treatments were plated in triplicate for a total of 21 wells used. Plates were placed in the incubator at 37° C. and 5-10% CO₂. Plate was placed in the incubator at 37° C. and 5-10% CO₂. Plate was removed every 48 hours and representative images taken with an Andor Zyla 4.2 sCMOS camera coupled to an Olympus IX71 inverted microscope. Plate was removed for maximum 1 hour before returning to the incubator. Experiment was concluded at six days.

### Example 11: Colony Formation Assay

The ability of compounds 1, 7A, and 7B to prevent colony formation was assessed in SUM159, a Triple Negative Breast Cancer cell line. Cells were grown to 80% confluency in T75 flasks in DMEM supplemented with 10% FBS, 1% amphotericin, 1% penicillin/streptomycin, and 5 μg/mL insulin. Media was discarded and trypsin added for 5 minutes to detach cells. Media was added to neutralize the trypsin and mixture was transferred to centrifuge tube. After centrifugation at 2000 RPM for 5 minutes, the pellet was washed with PBS and resuspended in 2 mL of above media. Cell density was assessed via hemocytometer and solution diluted to 2,000 cells/mL. Cells were plated in 6-well clear bottom plates at 2,000 cells/well. Treatment conditions included control (0.5% DMSO), and compounds 1, 7A, and 7B at both half and full IC₅. concentrations, All treatments were plated in triplicate for a total of 21 wells used. Plates were placed in the incubator at 37° C. and 5-10% CO₂ for six days. After incubation period, the cells were fixed with 4% paraformaldehyde for 20 minutes, stained with crystal violet for 15 minutes, and photographed.

### Example 12: Bioactivity

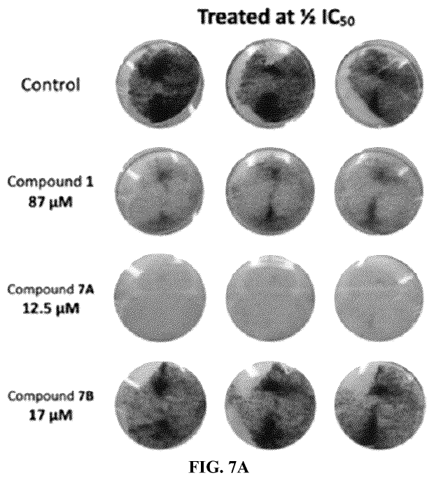

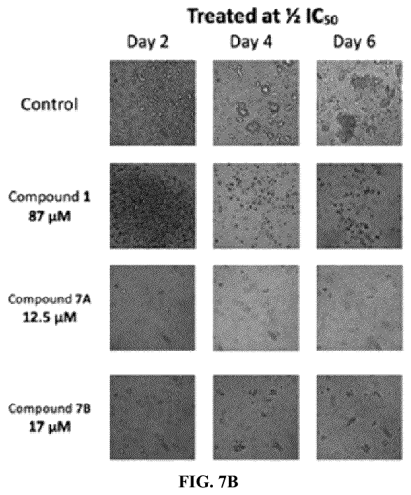

To further characterize the improved bioactivity of compounds 7A and 7B to compound 1, colonogenic and mammmosphere formation assays were performed in TNBC cell line SUM159 at full and half IC₅₀ values (FIG. 7A-7B). The results confirm that Machilin C and D (compound 1) possess the potential to slow colony formation and mammosphere growth. The unlimited division of cancer cells to form colonies characterizes its tumorigenic potential. Using colony formation assay, we evaluated the antiproliferative effects of 7A in SUM159 cells. We found that colony formation was inhibited across the 7A treatment groups at half the IC₅₀ concentration and the full IC₅₀ concentrations over a 6-day treatment period demonstrating potent antiproliferative effects of 7A compared to compound 1 as shown in FIG. 7A. Thus, supporting the development of unique analogs. Further, mammospheres are breast cells grown in a 3D-architecture to recapitulate the heterogeneity and pathology of breast cancer in patients. The translational potential of mammospheres is evidenced by resistance to therapy, enriched cancer stem cells (CSCs), and metastatic potential. We tested 7A at half or full IC₅₀ concentration and found effective inhibition of SUM159 mammosphere growth and a more enhanced inhibition by 7A compared to 1. Additionally, these results confirm the effect of diastereomeric configuration on biological activity, reaffirming that the threomer is the more potent isomer. This supports continuation of candidate optimization using the 7A structure as a new lead compound. Future research on this scaffold will delve into the enantiomeric aspect of the synthetic products. Current research has not investigated enantiomeric ratios in the natural distribution of Machilin C or D. However, it is hypothesized that the synthetic method produces all four enantiomers. It is necessary to isolate and investigate each enantiomer of lead compound 7A to confirm or nullify differences in potency, toxicity, and determine synthetic distribution. This remains an active area of research within our laboratory.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Gang, D. R., et al., Regiochemical control of monolignol radical coupling: a new paradigm for lignin and lignan biosynthesis. Chem Biol, 1999. 6(3): p. 143-51.
2. Mukhija, M., et al., Lignans: a versatile source of anticancer drugs. Beni Suef Univ J Basic Appl Sci, 2022. 11(1): p. 76.
3. Xu, X. Y., et al., Plant-derived lignans as potential antiviral agents: a systematic review. Phytochem Rev, 2022. 21(1): p. 239-289.
4. Dias, D. A., S. Urban, and U. Roessner, A historical overview of natural products in drug discovery. Metabolites, 2012. 2(2): p. 303-36.
5. Dzobo, K., The Role of Natural Products as Sources of Therapeutic Agents for Innovative Drug Discovery. Comprehensive Pharmacology, 2022: p. 408-22.
6. Atanasov, A. G., et al., Natural products in drug discovery: advances and opportunities. Nature Reviews Drug Discovery, 2021. 20(3): p. 200-216.
7. Chen, X., et al., Target identification of natural medicine with chemical proteomics approach: probe synthesis, target fishing and protein identification. Signal Transduction and Targeted Therapy, 2020. 5(1): p. 72.
8. Cragg, G. M., et al., The taxol supply crisis. New NCI policies for handling the large-scale production of novel natural product anticancer and anti-HIV agents. J Nat Prod, 1993. 56(10): p. 1657-68.
9. Harrison, C., Patenting natural products just got harder. Nat Biotechnol, 2014. 32(5): p. 403-4.
10. Corson, T. W. and C. M. Crews, Molecular understanding and modern application of traditional medicines: triumphs and trials. Cell, 2007. 130(5): p. 769-74.
11. Yadav, N., R. Deshmukh, and R. Mazumder, A comprehensive review on the use of traditional Chinese medicine for cancer treatment. Pharmacological Research—Modem Chinese Medicine, 2024. 11: p. 100423.
12. Zhang, X., et al., The positive role of traditional Chinese medicine as an adjunctive therapy for cancer. Biosci Trends, 2021. 15(5): p. 283-298.
13. Chung B. S., S. M. G., Dictionary of Korean Folk Medicine. 1990, Seoul: Young Lim Sa.
14. Zhen, X., et al., Machilin D, a Lignin Derived from Saururus chinensis, Suppresses Breast Cancer Stem Cells and Inhibits NF-κB Signaling. Biomolecules, 2020. 10(2): p. 245.
15. Ahn, B.-T., et al., Low-Density Lipoprotein-Antioxidant Constituents of Saururus chinensis. Journal of Natural Products, 2001. 64(12): p. 1562-1564.
16. Jeong, H. J., et al., Inhibitory effects of Saururus chinensis and its components on stomach cancer cells. Phytomedicine, 2015. 22(2): p. 256-261.
17. Yoo, H.-J., et al., Anti-inflammatory, anti-angiogenic and anti-nociceptive activities of Saururus chinensis extract. Journal of Ethnopharmacology, 2008. 120(2): p. 282-286.
18. Shimomura, H., Y. Sashida, and M. Oohara, Lignans from Machilus thunbergii. Phytochemistry, 1987. 26(5): p. 1513-1515.
19. Chen, H., et al., Comparative pharmacokinetic study of six lignans in normal and diabetic rats after oral administration of Saururus chinensis extract by LC-MS/MS. Biomedical Chromatography, 2022. 36(2): p. e5253.
20. Cuong, T. D., et al., Potent Acetylcholinesterase Inhibitory Compounds from Myristica fragrans. Natural Product Communications, 2014. 9(4): p. 1934578X1400900418.
21. Oh, K.-S., et al., Cardiovascular Effects of Lignans Isolated from Saururus chinensis. Planta Med, 2008. 74(03): p. 233-238.
22. Kim, Y. S. R., Shi Yong; Yon, Gyu Hwan; Hong, Kyung-Sik; Lee, Byung-Hoi; Lee, Byung Ho; Oh, Kwang Seok; Seo, Ho Won; Seo, Jee Hee; Koh, Woo Suk; et al, Use of root extract of Saururus chinensis for manufacture of medicament for vasorelaxation, W.I.P. Organization, Editor. 2008, Korea Research Institute of Chemical Technology, Korea, Republic of
23. Ahn, B. T. B., Seong Hae; Jung, Tae Suk; Lee, Eun Suk; Lee, Sae Bom, Lignan compound having antioxidant activity to low density lipoprotein. 2001, Korea Research Institute of Bioscience and Biotechnology, Korea, Republic of: Korea.
24. Lee, W. S., et al., Antioxidant activities of a new lignan and a neolignan from Saururus chinensis. Bioorganic & Medicinal Chemistry Letters, 2004. 14(22): p. 5623-5628.
25. Yoo, S.-R., et al. Anti-Inflamatory Activity of Neolignan Compound Isolated from the Roots of Saururus chinensis. Plants, 2020. 9, DOI: 10.3390/plants9080932.
26. Yun, H.-M., et al., Machilin D Promotes Apoptosis and Autophagy, and Inhibits Necroptosis in Human Oral Squamous Cell Carcinoma Cells. International Journal of Molecular Sciences, 2023. 24(5): p. 4576.
27. Jin, Q., et al., Lignans from Saururus chinensis with Inhibitory Effects on Nitric Oxide Production. Journal of Natural Products, 2019. 82(11): p. 3002-3009.
28. Cuong, T. D., et al., Inhibitory effect on NO production of phenolic compounds from Myristica fragrans. Bioorganic & Medicinal Chemistry Letters, 2011. 21(22): p. 6884-6887.
29. Lee, Y.-K., et al., Inhibition of DNA topoisomerases I and II and cytotoxicity by lignans from Saururus chinensis. Archives of Pharmacal Research, 2009. 32(10): p. 1409-1415.
30. Aysola, K., et al., Triple Negative Breast Cancer—An Overview. Hereditary Genet, 2013. 2013(Suppl 2).
31. Mercogliano, M. F., et al., Emerging Targeted Therapies for HER2-Positive Breast Cancer. Cancers (Basel), 2023. 15(7).
32. Trabert, B., et al., Progesterone and Breast Cancer. Endocr Rev, 2020. 41(2): p. 320-44.
33. Patel, R., et al., An emerging generation of endocrine therapies in breast cancer: a clinical perspective. npj Breast Cancer, 2023. 9(1): p. 20.
34. Obidiro, O., G. Battogtokh, and E. O. Akala, Triple Negative Breast Cancer Treatment Options and Limitations: Future Outlook. Pharmaceutics, 2023. 15(7).
35. Zagami, P. and L. A. Carey, Triple negative breast cancer: Pitfalls and progress. npj Breast Cancer, 2022. 8(1): p. 95.
36. Werther, J., Saururus chinensis (4749142278), in Distributed under Creative Commons Attribution-Share Alike 2.0 Generic. 2010.
37. [symbol] , Machilus thunbergii in Hiroshima Castle.jpg, in Distributed under CC BY-SA 4.0. 2017.
38. Cao, G. Y., et al., New neolignans from the seeds of Myristica fragrans that inhibit nitric oxide production. Food Chem, 2015. 173: p. 231-7.
39. Xia, Y. and W. Wang, Asymmetric synthesis of machilin C and its analogue. Chemical Papers, 2010. 64(5): p. 630-636.
40. Toxicology and carcinogenesis studies of isoeugenol (CAS No. 97-54-1) in F344/N rats and B6C3F1 mice (gavage studies). Natl Toxicol Program Tech Rep Ser, 2010(551): p. 1-178.
41. al, F.K.-G.e., Ullmann's Encyclopedia of Industrial Chemistry 7th ed. (1999-2012). Flavors and Fragrances., New York, NY: John Wiley & Sons.
42. Lewis, R. J. S., Hawley's Condensed Chemical Dictionary 15th Edition. 2007, New York, NY: John Wiley & Sons, Inc.
43. Guo, Y., et al., Structure—and computational-aided engineering of an oxidase to produce isoeugenol from a lignin-derived compound. Nature Communications, 2022. 13(1): p. 7195.
44. Leopold, B., Hassel O., Lund E. W., Aromatic Keto- and Hydroxy-polyethers as Lignin Models. Acta Chemica Scandinavica, 1950. 4: p. 1523-1537
45. Mardatillah, A., M. Mutakin, and J. Levita, FeCl3-catalyzed Synthesis of Dehydrodiisoeugenol. International Journal of Chemistry, 2015. 8: p. 1.
46. Godinez-Chaparro, B., et al., Synthesis and Biological Activities of Dehydrodiisoeugenol: A Review. Pharmaceuticals (Basel), 2022. 15(11).
47. Sederoff, R. R., et al., Unexpected variation in lignin. Current Opinion in Plant Biology, 1999. 2(2): p. 145-152.
48. Bitchagno, G. T. M., et al., Demystifying racemic natural products in the homochiral world. Nature Reviews Chemistry, 2022. 6(11): p. 806-822.
49. Wu, Z., et al., Enantiomeric Lignans and Neolignans from Phyllanthus glaucus: Enantioseparation and Their Absolute Configurations. Scientific Reports, 2016. 6(1): p. 24809.
50. Lee, Y. and S. Q. Choi, Quantitative analysis for lipophilic drug transport through a model lipid membrane with membrane retention. Eur J Pharm Sci, 2019.134: p. 176-184.
51. Ralph, J. and R. F. Helm, Rapid proton NMR method for determination of threo:erythro ratios in lignin model compounds and examination of reduction stereochemistry. Journal of Agricultural and Food Chemistry, 1991. 39(4): p. 705-709.
52. Asare, S. O., K. R. Dean, and B. C. Lynn, The study of the chromatographic behavior and a mass spectrometric approach to differentiating the diastereomer pair of the β-O-4 lignin dimer series. Anal Bioanal Chem, 2021. 413(15): p. 4037-4048.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A compound of the formula: 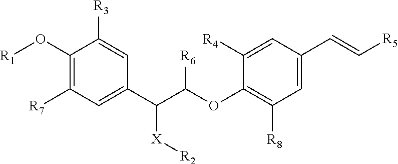
wherein
X is selected from the group consisting of O, NH, and S;
R₁ is selected from the group consisting of H, lower alkyl, and acetyl;
R₂ is selected from the group consisting of H, lower alkyl, benzyl, ethyl 2-hydroxy, cyclohexyl, methylcyclohexyl and N,N-dimethylaminoethyl, so long as R₂ is not H when X is O;
R₃ is selected from the group consisting of H, methoxy, fluorine, and cyano;
R₄ is selected from the group consisting of H, hydroxyl, methoxy, fluorine, and cyano;
R₅ is selected from the group consisting of H, methyl, hydroxyl, hydroxymethyl, methoxy, methoxymethyl, and acetate;
R₆ is selected from the group consisting of methyl, hydroxyl, hydroxymethyl, and methoxymethyl;
R₇ is selected from the group consisting of H, hydroxyl, and methoxy; and
R₅ is selected from the group consisting of H, hydroxyl, and methoxy.

2. The compound of claim 1, selected from:

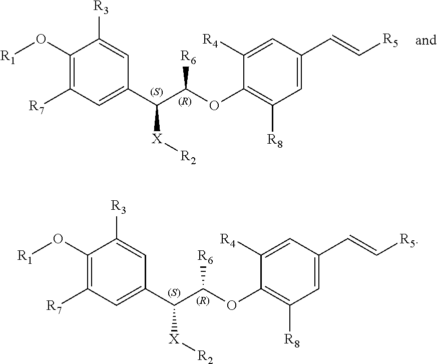

3. The compound of claim 1, selected from:

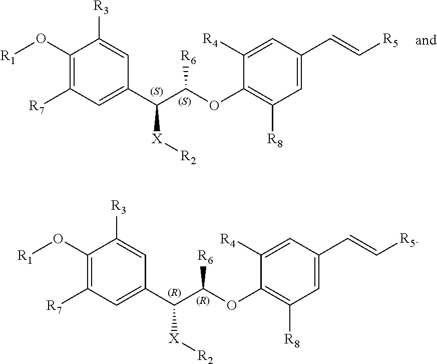

4. The compound of claim 1, selected from:

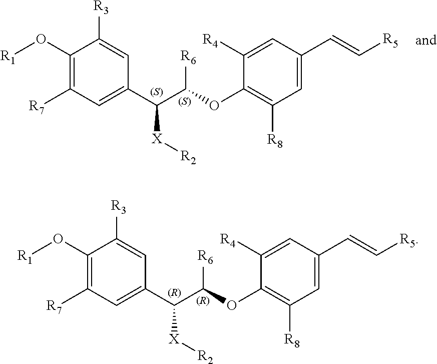

5. The compound of claim 1, selected from:

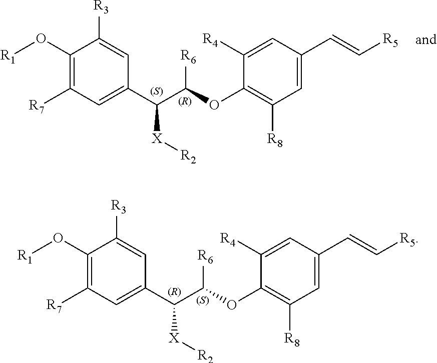

6. The compound of claim 1, wherein X is O and R₂ is methyl, ethyl, propyl, butyl, isopropyl, isobutyl, or isopentyl.

7. The compound of claim 1, wherein X is NH and R₂ is H, methyl, ethyl, propyl, butyl, isopropyl, isobutyl, or isopentyl.

8. The compound of claim 1, wherein X is S and R₂ is H, methyl, ethyl, propyl, butyl, isopropyl, isobutyl, or isopentyl.

9. The compound of claim 1, selected from:

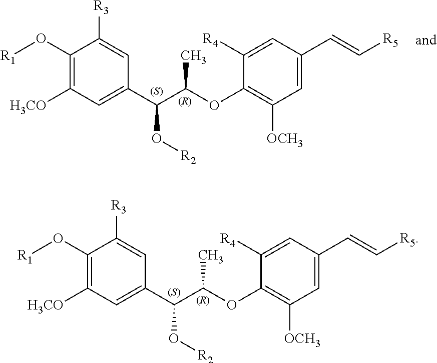

10. The compound of claim 1, selected from:

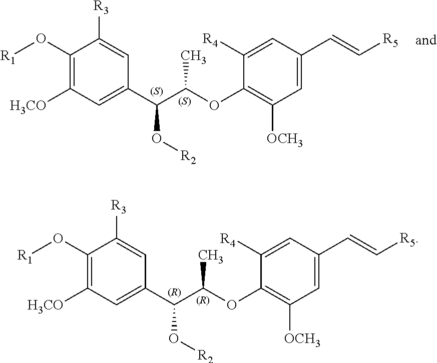

11. The compound of claim 1, selected from:

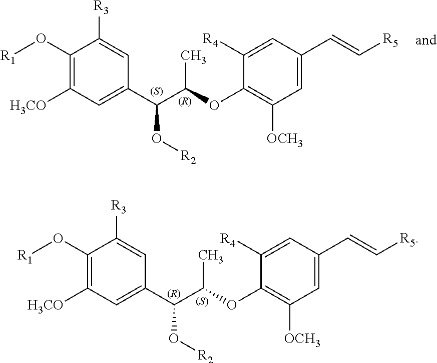

12. The compound of claim 1, selected from:

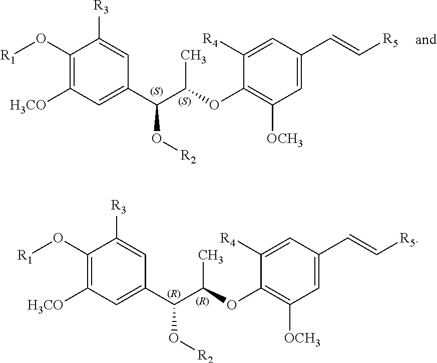

13. The compound of claim 1, selected from the group consisting of:

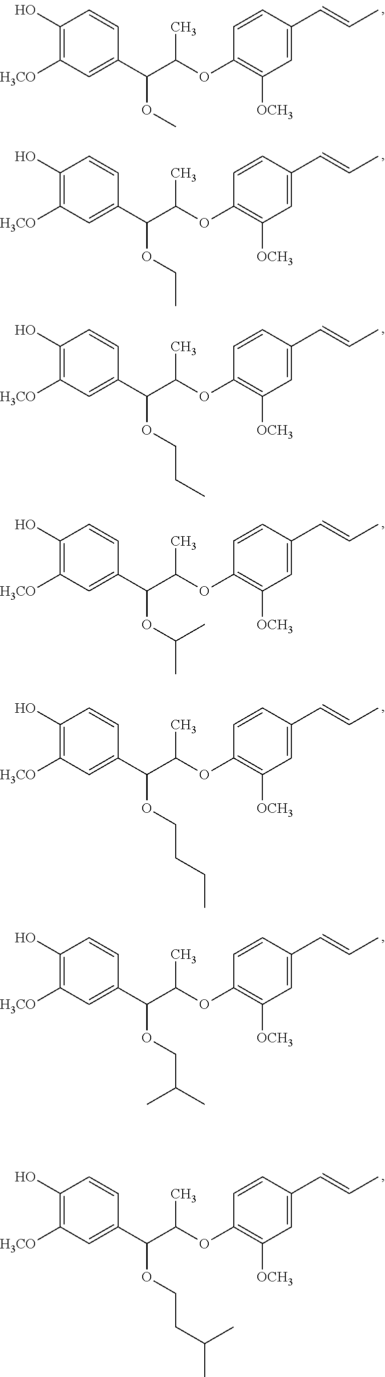

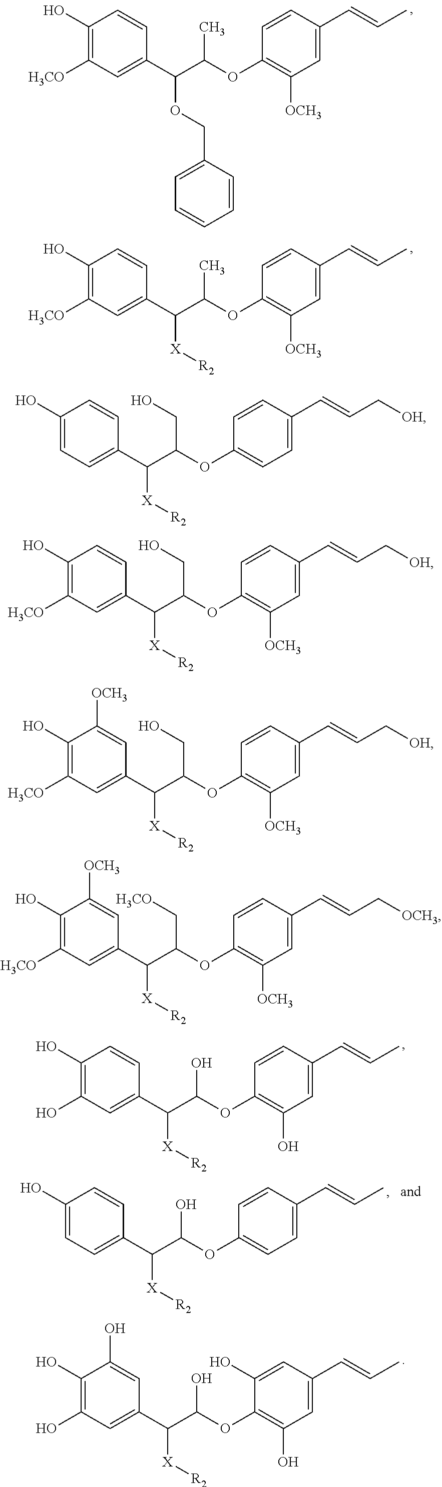

14. A composition comprising the compound of claim 1 and a pharmaceutically-acceptable carrier.

15. A method of conferring anti-cancer activity to a cancer cell, comprising: contacting the cancer cell with an effective amount of the compound of claim 1.

16. The method of claim 15, wherein the conferring anti-cancer activity results in one or more of inhibiting proliferation of the cancer cell, inhibiting metastasis, and killing the cancer cell.

17. The method of claim 15, wherein the cell is a cultured cell.

18. The method of claim 15, wherein the cell is in a mammal.

19. The method of claim 25, and further comprising administering at least one additional component selected from the group consisting of: cytostatics, PARP inhibitors, and immune checkpoint inhibitors.

20. A method of making a compound of claim 1, comprising dimerizing isoeugenol, an isoeugenol analog, or a monolignol in a reaction with a compound having the structure of R₂OH, wherein R₂ is selected from the group consisting of H, lower alkyl, benzyl, ethyl 2-hydroxy, cyclohexyl, methylcyclohexyl and N,N-dimethylaminoethyl.
